SEQUENCE LISTING (12 sequences)

SEQ ID NO: 1 - DNA, 3380 bp, Homo sapiens
ggacctgcaagcgccccacccacgccggggccgcagtaaccccgcgccgctctgcgcacgcgcggcttcaggctgtccgcggagctccttgagcccgcgtgggaaacctgggcgcttcgcgcggagacgcctttgtggctgcttcctggagcatttttttccccttagagctgttcgctgttttcctgtcacgccgcttttctgaaagacgggttgtgcgctttgggcttattcctggacggtccccaaaggtccccgaaaggaccaggcccggacacgtggggccttggtttccggccggaagtcccgggaggtgtagggctagagttctggccgtggcgggccggtttctgcgtgctgcgtgcgcggccgcgtgggctatggggcggcggtcgcggggtcggcggctccagcaacagcagcggccggaggacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaagccacgcaaaagagattctccattgcttaaagaacaaatattttaaggaattggaggacctggaggtggacggtcagaaagttgaagttccacagccactgagttggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattgaggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacgggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtgagcaggcccaaggcagcgggggcccacacccctcacacgcaaaactggcttcttctggtcactggtgtctgaaaccaaatccagagcagcctgtggcctgtaaagcatatatttctaatgactgcagactggtgggatcataggagccttctgaatgaccaggactgctttctttggagctgatgaaaatgtactcttttagcgtgttagaaatcacttgttttattttgtttctttggccaagctgggtctagtgtttcttttgctgggaatagactttcaaaagttgtacttctatcaagaaacaaaactgcccttgcagaaatttcaggtcttttgttaagcctgtattggtcttaaggtgcagtattttttaaattattatttatagaaagaatctataaattcttggggaagtgtgttataagctttaataattacattgagctgcacctcagtggtgtgtcattaacatgcagtggggttaatatctgaggcctcagatgactttgtgccttttggaataaagggtaaaataaactctcccagagtaagagctgtatcgtgaattgtcatactaattattgagggggacttatgtgcttttattgaatggagtgctttacaatttttatttttaaatggggttgggatccttggaatatttcaataaaattgataaaatataaaaaaaaaa

SEQ ID NO: 2 - protein, 767 aa, Homo sapiens [sequence omitted]

SEQ ID NO: 3 - DNA, 33806 bp, Homo sapiens
gtagggctagagttctggccgtggcgggccggtttctgcgtgctgcgtgcgcggccgcgtgggctatggggcggcggtcgcggggtcggcggctccagcaacagcagcggccggaggacgcggaggatggcgccgagggtggtggaaagcgcggcgaggcggtaggagccgggacccggcgggcgggccaggggctcctcctgggcttcccggaccccgaggcctgcgaggcggcggcgagggggcggctccttgggcctcggggtagacgtctctttccttcctcagggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaaggtagggaggcagtgcctgattttttgggagttggggccaggatgctggaggttaaaggcgacggtgttttcttcgacaagtgttacagtgccagagaaccgtggcgtttcagcggtttggattcaggaagcattgaggtggggagcacagggtccaatgaaatggtcacggaggtattgcaggcgcaaggttagaagacgaaatgaaaatgagatctgaggaagattcggctcctggggaatcttccaggagggaagctgagggtcttaccttttggaacgtaagagaatttatagagaatgcataaaaccccttggttctcccctcctccgcgccccccctccatctttgtagttacatctttctaaatgagtcgcaagtaaaacttaacggtaggatatgcttaggagagaaggaatgcagtgttgctcttagggctcaaacagtgtggattgcttaaattttgaaggcatacagtgtaaaagtagtgactaaagaatgtttactctccatgcaaaagttggttttagttttaaaacacttgcagtcttaaatttaagaagatacaatgtattaacttagttttttagatcagtttgaaacttccttatttttcaatagccacgcaaaagagattctccattgcttaaagaacaaatattttaaggaattggaggacctggaggtggacggtcagaaagttgaagttccacagccactgagttggtaggtaccacagtgcttcatgccgaatatttatttgggatattaatctctatcacttgaattatatttcttggtactgcatttgctgttaaagagcttgggtatactttgcaaacatcctaatgttctagtacctggtgcttttatcaacattatattgctacgcaagttttcatgcgatttgcgatttgcagctttatgagtcactgatttcagccagctcttcctagtacatccagcctcttcctttatgtcaccacttccgccagctcccttttacagcccttgttttctcacatcaaaactgttgcagagtggctgctttgccttcagaaaaaggacagacttagcttgacaacagtctcctggtaggtagtctcttagtgttttccattacagacaggttaccactgtctgctgtcaggttgtctcctggtacttcgcagtacagactgcaccagccaatgtagcatttccgagtctcagcacccaagcgtgctactactctgggagctacctctttcttgacctgtcctggaggtgccttctgggttacctattcctcatggctcggttctagtctcagttgtatgaaggtgatcccttgtgctgatagcatcttgtaaagatgcttggagaactccggaggcgccagtccttcagttaacttacataagtgggaaagtcgttattattaactttgaggtattgagttctcagaaaagtaatgcacgaaaaggtgaattaagccgttttttgtttgtctgtttgtttgtttggagacggagtctcactctgtcgcccaggctggagagcagtggcaccatcttggctcactgcaagctccacctccctggttcacgccattctcttgtctcagcctcccgagtagctgggactacaggtgcccgccaccacgcccggccaatttttttgtatttttagtggagacgggtttttaccttgctagccaggatggtctcatctcctgacctcatgatccgcccacctcggcctcccaaagtgctgggattacaggcgtgagccactgcgccctgccaaattaagcgttttgcatcaacttttgaggttttgactattccagtacgtagtgttatctttcctttttggataacctttagtgttcgcaaatcaaggtcagattttcattaatcatggggtacagatttgcttgtttttctttcattggctatttggattttcattgtccctatcacagataagcatatacattcacaatgaatgctgactggccaaacattaagtcagtattcaaccagtatcaagaccgaatatcaccatcatggaatgtgttgctgggatataggaataaatatgaccaacatcgtgaaaccctatctctactaaaaatacaaaaattagacaggcatggtggcacacgcctgtaatcccagacactagggaggctgaggcatgagaatcgcttgaacccaggaggtggaggttgcagtaagccaagatcatgccactgcactccagcctgggccacagagcgagaccatgtctcaaaaagggaaaaaagaatagatatgccacatcttagatttttaaaagcttgaagggtacagtataggaaaatgatacgggtgctacgttgctgttagtggtcactaattgtccactgtagtgagatcagtggaaaaggtagatgtttttcagcctctagttatggcattgctgagatgaggtaaccttcacgagatcaagcttttcttcctcctggtaactaggttgtagactttgctcaagtcgtttggtttgaagatttgattgacgacactgattgtgttgtacttgaagtagaggtctgatactgtagtagtccattttcacactgttataaggacatacctgagtaatgtatagataaaggaaataggcttaattgactcacatttccacatcgccggggaggcctcaggaagcttaaaatcatggcggaagatctctctgcacagggtggcaggagagagaatgagtgctgagcaaaggaagaagcccctaataaaccatcagatctccccctgagaacgaactcactgttgtgagaacaacatgagggaaacggcccggggatttaattatctccacctcgtcctgcccttgacacgtggggaatattacaattcaaggtgaaattggggtgggaacacagagccaaaccatctaaaatattttaagaagacaggattacgattctcctcgggcatgtttgtatttttagcagtgatgcatgtatgtgctgcttcacacaatcacctggaaacatttgtttctaaaaccttgtgtttagaagcgcagagacgggataagaaatgcttttaagatgaagaagttcctgtccaggaggaacgtttcagcatgtgtctatagggaagaggtgtgctcttaaggcacctccatgtgggcattttcttctgcaggctccaacaaactaggagaaaccatacgatgtcaataaggcttgctagttttttattattctctgcaaactacagttacacttggatttgatcagcccttttaacatttcatagctgcttttccaccttgtttaacaatgtgtctgggtatatgacaaggtaattaccaaactcttagaatctgcaggttgtctgtagttgaagaatgaaggtgaattgctctctgccaacagccagctatgtttttgtttgctttaattttctgaacacacagctgaagggtatgaacaaaacattgtagatctgggtttctaatatttggctttttccattatcaatgatggtttctctatattttattttgagacagggtctcactctgtcacccaggctggagtgcagtgatgtcatgacggttcactgcagccttgacctcctaggctcaagtgatcctcccacctcagcctcctgagtagctcggactacaggtgcatgccaccacgcctggctaattttttttatttttttgtagagatggagtttcaccttgttgcccaggctggtcttgcattccagggctcaagcaatcttcctgccttggtttcccaaagtgctgggattacagacatgagccaccattcctggccaatgccagtttcttggcagctgtattctctaaacatttatttattcaaattcatctgtcagttttagggtactaaatctctcagcaagcaagtaaagcagaagctttccaggacttctggtaattttctgtattgctcaagatgcaggaatagatcatgtaatattcttcccaatcctctggtacatagtgaaatataatttaggaaaacgttttttgaaattacattttctagaaggaaagttatgtataggaccatagcttgggccagtttcatttttttcttactttccatagactcagaacaatgagaaattatcctaaagcattcttatagaagcattgttttgacaaccaattgttcagagtgcaccttattatcattctatttatgtgctgtttacttatatttttttttgagacaaggtctctgttgcccaggttggagtgcagtggcatgatctcagctcactgcaacctccacctcctgggctcaggtgatcctcccacctcagcgtcccaggtagctgggattacatgcccccatcaccatacccagctattttttctattttttgtacagttggggttttgccatgttggacaggctagtcttgaactcctgacctcaagtgatccgcccaccttggtctcccaaagtgctggtgttacaggcatgagccactgcacctcgcctgttacgtgctttttaatgtctagattgaggatggtttgtcatagatgttgtatatatttacagagtccttaccacttgagaatttttaatatgcaaagatgacagtggtccttttaacttatcctctaaaaagcccaaataggattttttcttaaaagacacaagctaaatttgaagtaagtaactaaaaattaaatatacctctcattttgtttgatgttaaaatctacaaagatttaataaacattttttggttcataaaaagatgatgctgttcctaaagctgtataagaaacataatttaaaaacatttcttgtagaaacagggcctcgctttgttgcccaggctgttctctgtacccagaggaaataaacatttatctgttagtttctgtttgtatttaaatatttcagagttcctaaatgtttctttacatgagatcaacagccttcacaattataaatactgtagaattttaatcttgtaatagtacgtttacattttgactatcagaaatttgacgaaaaagacatataaggctatatttgttcaaagtatatctagactgcttatctatatttgaaaatgaaaattcagctctttacaataagtctaaatggcatgtggtgttcatattgatgtcttagtgatttggtaaggcaataacttacatgatcgtactagctttgagacagacaagttcaggaagaccagatactatatgagctggcatttggctgtttgcaagtagaggttacagtggcacagcagatatttgcccaatactatagcaaatgccaaatttgcatgtaacctactatttcgcctctaatgagtccaaaagattatttaaaacaattctttttaaaattgccttatgaatatgattttcttttggctttatatttttgaacacattaaatgtcgaggagggaactgggctatatgagatagaatcaaagcgctggggacctttggagggtggccgtggacagattcagccagtttcgtcttcattatttggtagaaaatgcttttcaggtatattttgtaatattttgacttgagcagtgtcgggcttttaagtcctgagtttgtttccatcaaaaaatcagagtttaaaagcagtcaaatgtgtttgtgttttccagacattccagttttattttcatggttttcactctagctacaatttgacgcatggaaacattgtgagcctgcattgttaaattgagtgtattactgcatctcaggtttagtgagaattgctgaaactacactaagatttatggccgtgtgacgtggtacacttggtttggcattggattgagcatggtgctgcatgacatcagtgtgctcatttgtacggtgcagatgcctgtataggtgcaggccaagtttcaaagcacatcctgggtggggtacagtggctcatgcctgtaatcccaacactttgggagactgatgcggccggatcactcgaggccaggagttcgagaccaagctggccaacacggcaaaaccacatctctactgaaaatacacgtctgtaatcccagctactcaggtggctgactgaggcatgagaattacttgaatccaggaggcagaagtcacagtgagctgagattacaccactacactccagcctaggtgacagtgagactgtctcaaaaaaaaaaaggcagtgtttttagtatcccccatttctcccctaattaaaaaaaataaaaaaaataaaaataacattgatgtcatcatttatagaaagttcaaatatattcacagatatgatggcaggaaatgctttattcttagttatatataggtaacagcctaggagctcaagttaatgcattagtggtttccaaggtaggatttttttggtatgtaaaatattttagtgcattattgaactggtgttagaaataatgaaaaatatattttttaaaaaactaatcaggtatttgtgggacactctgacactgcattattttgattctgtgtgatctgcacattaagtaagtcatcttgtatcttttttttctgatgaccttaccattgttaccttataatggtgaagagcagaggagtgtccgctttttcatctgatctcgagcgtttcatcattataattttacagtaatatgcataatcttgtaacccccatggctctcctgtttctgattgggccacggtgtagctagagccatgttccttaagcctacagcgacatctctgagtgttgctgactgtccgtagaggattgtgaataggagcagggagaacaacattcgaagtctcatttggcatgcgactgatagaagcacaaagttgtcaacagacgactttagtatttataatccataaatgttttgctttctttttggtcaggtatcctgaagaacttgcctggcacacaaatttaagtcgaaaaatcttgagaaaatcgccacacttggaaaagtttcatcagtttctagttagtgaaacagaatctgtaagttgatttctttagacttgctagtttccttaaagctgtaaatgcatagatgtagtgtagttctgtttttaggttcttgtcactagaagcagtaggtggacatcacacacagtaaagacaactgcagtgtgtgcaccacctgtgatttctgattatttgtatatccgttgccttgtttggtttatctagtcatgaaatttagtaaggatatcgatgccagctagttaatttcttgtttcctcttgcttgtgtatttggatggctatagggttggggtgagcactgcactctcatttccttcttaggtacacatttcagatgcctgttgtacataaaaagtagtatgtttttaaggccatcttggtagggaataggtttttggttctgttttttaaaattgatttattttactaaaaatgaagcttttgattttactttttactgtgttttttttttttcttaccacacattgctcttggtaaccacaaaacacctaagcaccacaaaacccctaagcacagctttggtcctcacaggacggcatctgtcttcacattctccgtagccacccacattccttgagtgcagttaaggtgctgagctttttacctttagcatcagagcatccccagcaaaacagctatttgcttatgtactaaataattcaatgaatgcccataaagtttgggtattttttttggaattcttagtctcacatttaaaatcagaggtttcgtccttcctattttctaaactactaccttctagtaaatctgtgtgtcaccagattatggttctttttcactttggccttctgtcatctaagtaaccgactaaactgggcttgtgtctgcctggcttttccttcctgaatagtaacaagaaccaggaactaagtgctgccacataaccccatttcgtacttgagaccagtgcacgaaaggattctcttgctcaactttcaatctagatggaaggccaaatagagatcagtaagctgctagagaacagctcaggtttgcacttggaagagagcgtggggtttgtggggtgtgatacacatggataggttggaagagccaggttttggcatgtttggaagatggaggagatgggagtttagggtgggtagcaaatgttgtagggaactagtataggtttttgattaggagtcagcgtgatgagagctcttgagatgaatggcaataacctctgggttatggaaagctggccacaaaacatctacacagggtggtcatgaatctggttactgtctgccgggatgtccccatcttacttaatgctttaatcaaaaccttggatggatacagaaggcaggcatatcaggatcacacgtataactcaaaagcttggaatcagtgtgcctagtgagtgaatcaccacagactcatgctggccagaaacccatgaggtgaaaattagcagccatcagtgtaaaagcctgtatttaaggctcttgacagtcacatgcctgagtttaaggtgagtgatggatgcctgggtggctgttcatttccagaagtcccagcttttgtagggttgtgttggagcagaagcctcagcttcaaggcgattaaagtagaggtgtggttgaggctagtgctgggcagggcaggaggattttgtctgcacctgaatagttctgccttgtaacagatgtcctggccgactagagaagacagagggcagggagtgtggtgaggaattttagatattgaaggtctaaagatggaaagatggaagcagtggttgatgtcttcatctctttgaaaggtagttatgagaaatagaaagtgggcatgggctatgtctgaaaatagtggaggactaagaaatagaattttaggcaagaaggaaaaaccatttgacagtcactttctgtcctaagttgatgctgtctgttgaaggtatttgtttaaatatttaatagcatttctgtctttgagagctgcccctctagcttctgtgctctactgctatggggagttggggctctgtaagcttcaagtcccttttctttcatggaagttggtattatacctgaatttctaatatttgaatggtatagcaaccatttgtccgctagtgtacagatccatctcatttagtgttctgtgtgatagtatgagttctctcattctgtataagattatgtgctgttttcctgttgcctttgaaacaatgccaaagattgaaattgtctgcggtttttttttttttcctaattgttgctgattttttttttttcctcaagggaaatattagtcgtcaagaagctgttagcatgatcccaccactgctcctcaacgtgcggcctcatcataaggtagcatatagcaactagaaacgtgggggcgggcagcttgttaccagtgtcatgcctgtttgttttgatgaaaccattttttcttaatataattagtcctttttttcttcttttcactttggatagagagtgatatgtacgtatcttagtttcttttttttttttttttcttttttgtcctacttaatttttatggtttagaaaattttctcccctttagttttactgatttgtttaccatatggattgtcttcagtgatggtgattggctaaattaggttgaaaatgatagtgaacatgtattgagagctcattgtatggcaagcattactctgtgctcttttttcttttcttttttttttttttgagatggagtttcactcttgttgtccaggctggagtgcagtggcgcgatcttggctcactgcaacctccgcctcctgagttcaggcaattctcctgcctcagtctcccaagtagccgggattacaggtgcgcaccaccacaccctgctaattttgtatttttagtagagacggggtttctccatgttggtcaggctggtctcgaactcccgacctcaggcgatccgcctgccttggccttccaaagtgctgggattacaggtgtgagccactgtgcctggccttgctctgtgttttttaggtgaattagttggctttatcattcacacaaccctctaagggcagatactgtcaccatctccatttttccagatgaggaaaaggaagcaaggagaagtacttgaactgaggttacggagctgttaactggctgagctgagatttgagtctaggtagctctgttccttagcattatgttttcatgattcttacttgttcatctctatatttagtctctctggcttacagtttcattctcctcccatcctctttggagactgtacctatatcattctgtgattttggggcagtgtagaatccctaaaagtaaattatgtcatgctatagactctgtaaagaagtatatttagatcttggactagaaattgaaaaactagaattgctttaaattaaatgtagaatatttggttttttaaaaacatgaaacagtttcttgctttgttaatagatcttagatatgtgtgcagcacctggctcaaagaccacacagttaattgaaatgctacatgccgacatgaatgtcccctttccaggtatgcattggacttgtgttcaaaatggtaggaatgccactttggcagattgtagacaaggaaaagactctaactttgcctgctaatttggctctaaagtctcctatgacaagcagttgggttttccgtctaccggaagtagaactggacatcttgcctaccaccgtcagtagtactcaaacaaacacactcataccatgggggagctttattttagaattatgtgtgttttctataaaaacgaatggcatttcttttttttttttttgagatggagtctcactctgttgcccaggctggagtgcagtggcgtgatgttggctcactgcaagttccacctcctgggttcacgccattctcctgcctcagcctctcgagtagctgggactacaggtgcccgccaccatgcccgggtaattttttgtatttttagtagagacggggtttcaccatgttagccaggatggtctcgatctcctgacctcgtgatccgcccgccttggcctcccaaagtgctgggattacaggtgtgagccaccacgcccggctgcagatggtgtttctttatcaagcctacttacagaagatagatttcactgtttgaatttttagtaacaatttcccctttcccccacctttgttcattattattactattattattattattattacttactgccttacatgaagggtagtagtgtgtagttaaaagctaccatcagactccctgggttcaaatcctaatcggcaagttacttacagtgttagacatcccatgtgaaatgggacgaatcagtatgtagcttgcgaggtgctcaggaaatgttagctgttatagtgacaccacgcccctttctcttaagtggaaacaaatacccatcgtgatgttttagagtcagctttcatctcgtcctatctttgttcaggcaaacttctctagttctgttttaataggcatatttgttaggtctgttttttgaaatcctcttttttacattgtttaaagataatgccttggctaaaaagcctgcttcacttttccctgtttttagttgttttctccacattggcagtaaagagccttggcgtcccagtagcagcaggttctcctttttgtattgtggatgttttgcatttcatactgttgtgaagagtggctttgatcatacatgttgttggtatatttgcctttttgctgggggtgtgagaagaaccagagatgagcagaggtacacccagtagacttcccagcctgcagagcctcccgggaagagcttccgtgttcaggtgcttggggccccaccctaggagcctgactcacagtcagagcagggtcccggcttgtgttcaggattttgaaacatttgtaaggtgattttgttgtttctacacctttctcctcatcttttttttttgtagttaatcgttactaataacagaaaagacatttttggcatggtaattggcacaaagtgaataattgttgaatagatgacttttgaggctttcaaaattcgagtgtccataaaatccatccagagccacctggttcctttttttgaaccacttaacgtaattctggaaaaccttgactgtgggtcttaagtttggtggattgctgcttctcactggctgacctttggaggtcgcatatttcaggatgtgattccacttaggctccatttcacctgacactgcaattctgtgccttcagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcaggtatttatcttattggccaaaaagcccaaatccactaaagaaatcaagtcaaatagagactaaaatgaagttgtgcatgaatatgacacaattgtacataagtcttggggtgcggagaaagatagcgtgagcctaaaccaaaagatggacacgtagatacgtcttaatgagaagcagcacagcagatgctggataaagaacagaaaggggtggttgcaatagcctgcagaaagataaagagaaaacaaaacaaaacagagaaaatacgtaagaggaaaatagaatcgtagactcttattgtggtgtgttttaggatgttattgcttggaacaatttgtgcaccccaactattttgtattagttcttccagtgagtacagtatataaaaaactttgtcataaaataatacgtaatcacattggattttatgttatttagttgaatgaaagaaatttcggagttcaagcccttaagaaattcattttgcttgtgtttttatacctttcttgagataatacatcaacattttagatatttagagtttagatataaaataatactcatggtataactgagtaacaagtttttttaaaaggaaagaaacaaaactaaagacatcatctgttccgttcccataggaggtgcacggatgatgatcccaggtgataatgacttctgaatgacataagtttgtcaagaccagaattcaaaagctgccacaaaacttgttagcacagaatgcaagctttagatacgataccacattatggaagaatgccattttgtttcagtaagtggcagagttactgctcatggcaaaactcagtatgaaacttgcagctggtggctctgaatatagctgagtgttaaaataatctggaaaaagccctctttaaagtaaaatttaaaatacatcatgaactaaagtttttttttcctctaggttgcaatttttcagtattccttggggtctcatgttttcctacccctgttttttgatcagaaagataactcgttccttgtatcatattctgagatctgagaacgcagtgaccttatgagcaagaggttgcaagctggggaaatttctgaaattgtggtgggatttctaccatgcaaacaactagtgtacagccaggaatgccattgcaaagtggggattacatctcatgggataattccatcctgcagacctagtccctcgtgtacacgcggctagaccatcaaggaaaatcagcatttacagagaaagccaacaaaaacaggcgttaccttctgggaaaacagggcagataaaattagacacagaaagtaacaaatccagtctttactataaaatatacatggaaagagcttccattaacaaaaacatgaaaataacttccattaaataaaagtagaaagtcagaaatttaacggtgattgtaagaaaaatttagctattagtaagaaaaatttagctattatattagcttaattgaaattactatggaaaatttattagtcattattcatataattctgattaaaatgccattgttgattaactgagtttttcaatagtattttaggttctgaaaaacatgaaaataacttccattaaataaaagtagaaagtcagaaatgtttacctgtgattgtaggaaaaatttagctaatatattagcttaactgtggaaaatttcttattagtcattattcatgtaattctgattaaagtgtcattgttgattaactgagattttcaatagtattttaggttctgtgtcctaggtctgcaaattagtcttgctagtgttttttccaaatggaagcacattaatatctccttagtgaatggagtttactgttcaaatccattattttctgcttgaaaattgaagtataatttaaaactataatacattagaaaatccctaaaacaaaagctgaaagcagattgcagttggtgaaaatggagattgtttcctaactgatatttgtaacacttgttttgtaacttagcttgtatgtgacatatctgttaaagtgcagtcatccacctgtagggagctttgcactcataaatatcttttttccagtggagacggcactatgagaaaaaacattgatgtttggaaaaagtggaccaccttaaatagcttgcagctacatgggtaagtaaaacttcatcactgctaacactgtgtgacaagtagatcagcagagatttattgttatgcaagtgagcatccgatctttaattagagagctgtaagcatcagaggatggtcaatggccaccttcaactgttttatcagaagttatttgaaaacctttcaagagcaataactgcaaattgtatacatttcagataacacgtttattaaaaatgccttctataaaaatttcttgcaacacttcagatatgtatttttccatgaaatgaccgtttacctcttgatgtatgcattactaatcttttctctctgtttttgttcttttgtatcatcgttttgctaagagacattttgatctcttagaaatatttttcggttggctttttatgatggggaactattcattaaagaacaattggaaaattcttaaagagaaatctcataacacgatgatcacgtctgttaatgtttggtgtgttgtattgtttttctgtttacagggattttttaagtactttttccaaatacttttgatcatgctgtctgtagagcagtgtattctttttttcatttgacatgacaaatgccatggtcttgcatgcaggactgtactgtgcctgagccacagaatactaattagcatccaccacaaccaactatagcaggaggcctcgtctccataaaatactgtctactgttttgtgacacagagacaagagttaggactggaatgtatgataccaatttttcatagaaaaaccacatatttaggaagtatggaggttttttaaatagtgaaatttcatctaatacattgaattctgttgttactgaactttatactctgctggttgacatattgggaaaggttaacagagcagtaagaatatttatataatacattttttttattaacatgaaacactttatataagtttactctaacttcatttagatccctggaaggttttttttaagtataattgtgttataaaagcttgtaatgaatatgtgacataaggaggtgcttctaatatgtgcacttcatggatacatttggccttgcagtcatctgatatctccccttagcttacagctgcggattgcaacacgcggggctgaacagctggctgaaggtggaaggatggtgtattccacgtgttcactaaaccctattgaggatgaagcagtcatagcatctttactggaaaaaagtgaaggtaagcacgcttgatggatcttttattaacagcatagggggcttttcatatgtgtatggtacaatacacataacgtgaggtttaccatcttaatcatttttgagcctgcagttcagtcccgttaggtatattcccattgttaatcaccaccaccatccatctccagaaatcttcaccttgcaaaactgaaactctgaactcctttaaacagtagctcccatctctctcttccctgcccctcacaccaccgttctattttttgtctgtatgactttgactactctagacctcacagaagcggaatcatactgtatttgtcttttcgtgactggcttatttaacttactgcagcgtccttgaggctcacttgtgttgttgtctgtgtcaaaattgcattgcttattaaggctgaacactattctgttgtttatataaaccgcattttgtttacccatccatctgttgatggacatctgggtttcttccactttttggctgttgtgagccatgctgctgtgagcatggtatacaaatacctctccaagatcctggttacgattctttggatatacactcagaagttgaatggctggatcatatgatcatatagtaattctgtgtttaattttggggggaccccaccatgctttttttcacagtggctgcgtcattttgtatttctacccccagtgcacaagggttccaatttctctacacattcagggacacttattatctggggctctttgttgttagtagccatcctaatgggtgtgaggtcacctcatacagttttatactgaggaactgacagatagtacagaatattctgaaacagtagtgtatttctacagtgtattgatataaaatatttctccttttatgttttgccagaatgttaacttttcccatcatattttccttagagccaataatgagaaatttcaaacttagcatgtttcctgcacccacgtgtgtgaagtcagttggtgcatagtcagtgagagaggcctttcctgggcagtgcccacctgctgctgtccttccagtgaggctccccttggcggctggctggtctgggttgtgccctcagccctgcggttgttgatggccttcacggtcctttaagctggaatgtaatgttgtgtttattttcctgtgctcatgtagacctctgtaaagatgtcatttctgggtgatgttttcttgcatggtcataattgccatggtgttggtgatactttattccaaaaggtgtgtgagggggagtatggggcacataatagtttagttgtaatgaaattacaccttcaagtgtgatttggggtcagttgttaggactgttgaatcctgctcagatttttgtgttcattagctaggaatttaggaaaccatgattccaggcagcttgagcagcacagacacctcctgtttcccttgccacagtctgctttcagatccctgagaaagaaaatggactgcatttattaatttgcatgcctagttgccatttacgactcagacttgagggtgggccccagttcctgggcgcactcactgcattcttggctggtgctctttgtcttttgtaaagacagcttcccccggtttctcggtgagcagctttcttgacttggggtgagtggtcacagccccatcttgtgctgcttagcttcaaatgtttgtagattctttaagcatttgctactctcctaagtctgtgttggctagttaagtactctaggtgtttaattttgctcctttttctcttgtaagcttttggctgggaccaaaagccaggtctcctgagttcttaggtgagagaggctcttgtccacacaagcctgtcctcactcgtggctggagtcactctttgcaggcaggacttgctggcctgagtccaccgggctttctttcctcacattggagatacttgctgtttgcctgtctcattaccgcaaaattgtccccgctcagagaacatgctgtgtgctctgtgggctggctgtgctgtcagaccgggttacttgcctggagctctggatgcttctgccaccctcacccatttcctgaacgtgccaacgtttgaaggagtggcttcgtgaggggttgtcatctgaaagcaggttgaatccaagttctagttctcattcccatgggtctctctttccagcagcagtggggaaccatcattctcctgaaaacccactgatctctaagggaagagtggacagaacctcacacacagtgcaccatgttcgaaagttaagaatgagcaagcgtgacccaggcacgggggagtgaggaagagctgcagggtgaaaaagaaaacgggaatgctggtgatcaggattgtgaggagggatgagcagatgtgggattgtcctggggtactgatgggttcgtcttcatccggtctgaaacctttcttgattgtggattttcattggtctttctgattttcagactggctggctggttctttttcttgtatttagaatgttactgctggttttctgagtgaacgcttgcagatcttttcagacttcaccaaatttgtggctctagttggctttatgtctgtctatatgtgtgtgttgtgggtttttttttttttttttttttttttttccgagacagtctcgctctgttgcccaggctggagtgcagtggcgccatctcggctcactgcaacctccatctcccgggttaagtgattctcctgcctcagcctcccgagtagctgggactacaggcatgtgccaccacgcccagccaatttttgtatttttagtagagacggggtttcaccatgttggccaggctggtctcaaactcctgacttcaggcagtcttcccacctcggcctcccaaagtgctgggattacaggtgtgagccaccatgcctggcctggcaggctttcttttgaaagcccagtaaataacaccctctttccctgtccagcttctgaaccaattgacaaggtaatggagcaaggcagaccaaggccaaaatcgcacctttattaccaatagagcagatgatatgctttcagatacttacctttaaaatgaggcttatctcctcattgtcacaggtcacagaaggtgttagactatggcatgcagttgagctttgttctaagggcaagcttcactgcaagacaagaaaggagacagtatgctcaggttctttattcaaattttggaatcattttagctttctgagatgccaggggagataccttgggggtggagggagttcttggtcaggggctggcttctgcctccacataggagatttcaaagaacagccagcaccgtttggccagtgggttttaagtataagggcattcatatttagaaaaaaagtacatggattcttttccttcaagtaactgcattgctttttgcaacttttcctctagtcttcactgtgttgaatagttttgattgtgttgtgtatatcttaaataccctgctttttcacctaacattccaatatgtcatagtagtttttcctgttagcaagtgtcaaaaatgaattacttcattcagtgagtctcctattctgtagtcattttcttactggacaggtgggtttccagcatggacctgtgtacagtatgatgtcacagccaactttttgcatgtgggtggcttcagaccgtattgtagatgcttcctcccttttctctgtcagaggcggtgtgcatggatatggctttatgcaggaaggagcagaccttggctgagccttagagtgggtggttgctggtgattttgactggagggggtgtgcgttccccacctgaaagtgctcaacaactttactggagaaggggcaacatggggtagcattggcagcttccaacccaggtgctggggtgtggagataacacctgctgcctgccctgctcatctgtgaagcccctggacagtgtttgatggagaagcccgcatgtttgcctgcactgtcatttactgttagcttttaaaaggaagaggattcagaaatgatggcagaagtcagtacgatgcttgcttcaaaggtttcttactgccattcctccaccttggagattcttttcctttgtttttgtaattgtcccattttcatgttttctgggagatttgttgaattttcctctgctgagaggctgtcacatcagtatctgtgctagtagctggtctcactggtttcctgtaatggcctcaccttatagcttcttattgtgtcactggtatggacacttaaggccagtgtgttttcacagctcttatcgcattgtttttaaaactgggctgactgtgtcccgatgggcaattttaggaaacttcctgggaccacgaacttgaggtttaaaaattcccatagttcattttcagcattgctctgggatgtttgttagacatgcctgcgtgaagacctgcagcatcgccctggtgtgcattggaatggggagacggagcaggcgtcctgcagcccagccttctggttgtcttcttgcggagttagtctcgcgtctgtggcaggactcctcaatgccctgtcattggaacaactttattgagctgtcgtttacatattgttaatgtttgctcattttaagtgtacggttcagtgatttttagtaagtttactcagttggcagcgccttaccacagtccagctttaggccgttcccgtcaccctagcagacctttcctgcccaacagcacttgctccccattccagctgagccccatcggccactcatccaccccgtcggccactcatccactttctgtcgacgggtttgccttccctggacattttctgtacatggaatcatataatatgtgatctgtgtctggtttttttcactgagcatagtgtgttaatacttttttgaaaaacaaaagacgtccatgcttttgctgcacaggtgctttggagcttgctgatgtgtctaatgaactgccagggctgaagtggatgcctggaatcacacagtggaaggtaacctttcctcgagaactttcattctaaagagtaggtgcagcatcactgaagtagagtcaagtttcaaagcattcacgtgtgagtaacttgaataaatactacatctggttatgccaattagaatcaatttcggagtgttatttcatgacacatttcatgacaagtggcatgtttattcctggcagtggaaaagtttttttttctccacgtacagaaataaactcttttactctcatccctgtaagggtagctttgctttttttttttttttttaaattgggccgggattcaagccttgtttccaatatgaagtaattcattacaattttaggccagaaacagcctgaggcttgtttaaaaagaaaaaaactagatggaaaatgttattttataatgcttgtcctggtttttagaataaatgtatttcatctttgtttttaacagaatttatgtattaataatttggggattttctgtaaagattgttttgttttgtcttggcaaataatcttcctatctttggagtgaatgagaatcaccatttgtcacctttgagagaatggatactcctgccctgtgatttttgttggtattggatagtgctagtaatctggaatgtaccctgtggttctgcaggtaatgacgaaagatgggcagtggtttacagactgggacgctgttcctcacagcagacacacccagatccgacctaccatgttccctccgaaggacccagaaaagctgcaggccatgcacctggagcgatggtgagctggtggggcctccaggtgtggggaggggggaaggttaagccccatccctggtgagctggtgagtcttgttggggtgagttggaggcagcaggtgacattgctgtgaggcttgggttatgccagggtcaaccccatgtgtgtgaacttcccagccagtatggctcttgtgcctgttgctggcattgagcatttattaatagtgaaattggctagaatctaaatggtaatgcttagggtacttcatgaactttttttttttttttttgagagagagtctcactctgtcatccaggctggagtgtagtagctcgatctcggctcactgcaacctctgcctcctgggttcaagcaattctcgtgcctcagcctcccaagtagctgggattacaggtgcatgcctggctaatttttgtatttttattagtagagacagggtttcaccacgttggccaggctggtgttgaactcccaacctcaggttatctgcccacctcggcctcccaaagtgctgggattacaggcatgagccactgtgcccagccgtcatgaactttttaaagattacaaagtgggccaggtgcagtggcccaatcctgtaatccccagcactttgggaggccaaggctgatggatcacttgagctcaaaagtttgagaccagcctggacgacatggcaagaccccatctctacagaaaaaaaaaaagaaaaatacaaaaactggccaggcagtgtgttgcatgcctgtagtcccagctccttgggaggctgaggcaggaggatctcctgagcccaggaggcggaggctacagtgggttgagatcacaccactgcactccagcctaggcaacagatccagaccttgactcaaaaaaaaaaaaaaaatttaagtttacatattggccattctatgagtgagggtcgaagacatgatttttctcttgtatcatctttgcggtatttgtcttaatagtaaaagaatgcatttttgattagttcagaggtgtgaattatatatatatatttagccttaggatattaccccatcatcagaatactggagggttttttgtggcagtattggtgaaaaaatcttcaatgccgtggaataaacgtcagccaaaggtgagtttttctttttccaaaatgacataacatttgatcttgtacatttaagacaaaaactaaccggagtttagtagaagtacagaggaaagaggagcttcttgctgtgggcagcaggagagctgaccctgaatgagggggaatttcatttaaatatcaagttttccaaaaagcagaaatttctcataggtgataggtaagtggagaagtcagtgtttgggggaatgtattcctgccacactgtaaatccagttatttaataaaaattgaaaagacatgaaagattgctctgtggctctcaattggaagccccaggtttctgtgctctagttccttgtgagtggttcatttcaccaattacagatagcagagctctgctgaccccaagccagcccgggttcaccttggctgcaaggaatgatgacggccttgtccagacctggctagaaagatgcagcccggcctgtttgctatggatctaaactgcctgctggttcctttccaaggcaggccaggaaacagtggtgaaggagtgttgccctcatcctaacacgcagtcctttgtaatgcgtgctgtctcacctgtatcacgccagcattatttattagttcataaatcagccttccatgatgaaagaacctggcctggaatcaaagtctggaagtctgtatttctttaagatccatgcttgaaaattaggacaaaaaacgcttagctttggaggaacaaaaaggaaacagttccgcaaagagctccagcctttttctggggcacggtttgtgcagtttaacgttggaacgtacagcctcagacgggcaaagggggcgactgcacttctgccgccaccagggtttttctgtcaggttagaaagtatttcactttgaggctaaaagtctcacaaggtatcttaacgctgatggaatgttattttcatggaatcagtataagaaattatattgtaaagtattagatactttgcattcattcatactaggtttcagtagcttgtgttttagactttgcgcttgtcacattttaagtggtcagtgaccacaggcttgtggctgcccagctgcagagcacagtgcagtcacagaggagcctgtcttagagacgcgtgctttaggttggcctgcattagggcttacattgatgtttctgacgtgttaatacttacatagaaaggttttgacattttttcaattagccccttatgtatagtcttactttttagaacaacttattgtcattttctcgtttaaataatatgaatacttctctctttctgttactatgtcagtttcttattcacttagttcaacagatctagtgctagcatggcctgtgctgctgttggtcctactgggaacgcaggtagagtctccatggtcaggatgccgtgttctgttgtggtgctgaggcctgtaagcacttggctagaagttaggccagggaagctcacactgaccttggtatttgaaggtcccagaatgaggttgttgaggtagaacagagatgggagaacatgcccttggagctggactcaacaagaaggcccctctgggggaagctgaggttggacaggaagggcgtgtgccctcactgacttgctcacaggcttggggttcatctgttttggtttttgcttttttacattatattaacatggaaataaaaggtgttccctgggatgctcccggcttctctgctcagtagctttgtggctctgagtaaaatgaacttgcctgtgttgaaatatcctaatttttaaacttacttcatagggactgaggaattacgacttttatcaattttgtgacctgttaaaatgttaaaaaggacatgtattttttaaagatctttaagtaaaacattttgctcattcccaaagccaaatttaaattataccatggccctaattcagaagttcattctttggcaggtgtttccttggtgcctggggcactctctctttctcccagttcctgtggcagtttgtccaggtgcccaagaacaattcataccctcctttctcgttatttatatacttgtctttttgcccctgccgggtattttagaaatcgtgcttggtgcactttgtgtccttcagtgtgccttacccagagcagatgcacgataagcatttttacacgagaacaagctggtggtgtaggcctctgctaaggaacaggctgtatatgctctttgtgggattaaggtagaatcagctttaactccaaagaaactgtccatgaattttgtttataatagcaagtagatttaaaatgacactttaaaaaaattctgttgtctttctctacatataatgctgtagaaaaatataactgatggattttgtgaacgtggtatttttaacttttcgtagcagcacaaagaatgctcctctttgtgtgtcaatgaaattgttctttatgacagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtgggtaagaaaagtggttatgtcttgatctaatacgctggtgtcttcacagtccttttggattaaatgggatcccagagccacttcttggtcggtttgagggggcagtacatgtgtggtatcaggcacatgcagtgtgagggctggactctgtggaagccggaaggtttcaacatctgccgcaaaccgttccatgttgcacagaactgacagaaaggaagaatgtgcttttggtattgaaggttaccacacattacagattgatttgtctcatcctgattccctttttagtcctcctccatcaaagaaaatgaagttatttggatttaaagaagatccatttgtatttattcctgaagatgacccattatttccacctattgagtaaggattcagcctttttaattattcatttaaagaaatttactatagagtatcaaatgtacaactgatcacatgtaaccattgttttgtatgtagttctgtctagctttttttttttttttaacctttttaactgcatattagagcaggatgaaactttagaggttactcaatcttttaatttaaggagaaagtaaacttttactttgtgaacatgatagataaaaaaaaactggaccgggcgcggtggctcacggctgtaatcccagcactttgggaggccgagacgggcggagcacgaggtcaggagattgagaccatcctggctgacacggtgaaaccccgtctctactaaaaatacaaaaaaaattagccgggcgtggtggcgggcacctgtagtcccagctactctggaggctgaggcaggagaatggcgtgaacctgggaggtggagcttgcagtgagccgagatcgcgccgctgcactccagcctgggcgacagagcgagactccgtctcaaaaaaaacaaaagaaaactggactgtgattatgaatctaaattagttgtgatcctgaacctaaattactcaattgagtatatagacgagtagccaaggttgtctagttcagtttctgtagaagaaatgaagagcagcatgggtgagggctaattagggatgacatagacagaatatagaggaaaagggtttaggacaagtctgacattcatctgtttcttattcattgaattttagaatctatttaccagggcggtcacttactgtcttttttaataactggtcctttgcatacatttgtaaaagtctattaaaaaataagtctcagccgggcacatcccagcacttggggaggctgaggcaggcagatcatgaggttaggagtttgagaccagtctgaccaatgtggtgaaaccccgtctttactaaaaatacaaaaaaaaattaggtgtggtggcgtgcacctataatcacagctactcaggctgaggcaggagaaccgcttcaacccgggaggcagaggttgcagtgagctgagactgcgccactgcactccagcttggaagacagagcaagactccatatcaaaaaaaaaaaaaaagtctcaaaagtagtgttatcaggtcattatcattggagtagcggctgagggagagaagctgcacttgttaatttaggtttctgatggagttgtgccccaaataaatgttacgtggaggtttcaggtggaagtggatgaagtttagaagctggagtccagagcagagcccagtgcccttgaaccacactgcagttttggggattgaattctggaggaggagaataagatgtttagaaacagtctaccagattttgaactcattgtggataaacaacaatgttgttatgtacaggaaattttatgctttggatccttcattcccaaggatgaatttgttaactcggactacagaagggaagaaaaggcagctctacatggtttctaaggagttgcggaatgtgctgctgaataacagtgagaagatgaaggtgccagccgcacagacgctgagtgctgtgtgatgcgggatgcgtgtggcttttcacagcggcttagatttcatttggagcagaccgtgacatctgccctccccacctgtagcgctggctgtgggtccctgagcctgccctttgatttcagagctgttgactgacacagggctgaatggttcctgccagagtcctgaatctggtggcctgtgtcttttctgtgccccattcccgcaggggggtgagccatggtgtagttctcacagctcgccctcctgggcagagaaggtactaaaggtgtagaggctgcgagtactgctgccccgacccttgggatttgtcctttgaggtacttcccttagaatttggccatttgggggcccttctttcatttgctttactgtcctccaaacgacgtaatcacaagttgtgagtatcagttcgttcttaaccggggtttctcggtgggtttctgcttctcctctcctttcccattccgtgacggttgctccatgtggcccggcatccttacaggagatggtcctgtcttcatctctctgtgtttcatcttatttttttacttacaggttattaacacggggatcaaagtctggtgtagaaataacagcggtgaagagtttgactgtgctttccggctggcacaggaggtaattttggaagtggatttgaacagcagtagccacagatgactgaagcagtcgggagtagatgggcttgctggtcagccagatggtctgagtggctcagggcttggctggcacctaccagccacttgaccctcacaggttatagagctctcccagagtgctggtttcccctcctaggggggggtgggttccacacctgtccaaggggaggggccatagtgagggctcgaccaccatagccgctaagagcatagcccctgagagcaggctgttgtcgttcatgaatgacatcatggtatctgttctctcattctctttattcacacagggaatatatacattgtatccatttattaactcaagaattattactgtatcaatggaagatgttaagatactgttgacccaggaaaatcccttttttagaaaactcagcagtgagacctacagtcaagcaaaggacctgggtgagtagttggaaatgcatagagacataacttgtattccctttataaggccagcaaaaatgcaggtggtgtgcgttggggcatatttatcaatcatagttttcagtttatttatggctacatcaatttccagagggttctgtagatccctgattgacagttcagaatgctaagggtcaaaggcatcttggggttggtgtacttccccaggctctggtggtgccctgctgctgcccagcagcctgtggtgtgtctgtgtggcgtgcagggctggcctttgctcaccttgctgggccagagagtccggggagaggaggggtcagcctgtgagcagccctggcaagcctcccagccagagctcttgacccacttgcctgtcagcaggtcgggggtgggtggaagcaggtctctgtgcatgagcaaatttgtttttttctttttttgttttgagacagagtctcgctctgtcacccaggctggagtacagtggcacgatctcggctcactgcaagctccgcctcccaggttcagccattctcctgccccagcctcccaagtagctgggactacaagcacccgccaccacgcccagctaattttttgtatttttagtagagacggggtttcaccgtgttagccaggatggtctcaatctcctgaccttgtgatctgcctgtctcagcctcccaaagtgctgggattacaggcgtgggccaccgcgcccagccagtaaatttgttttttaaggtacacagcctcacgagactggctaacatgcagattttaatcacctgtgttagagcaaaataagagtcttgccagcagttctgacttttctgctagccagctggagtagattattggtcatattgctgcttttatatttcatcaacttccacttcagatcactccagtgacgttttcccctaagcataaaaatcatttctccaagctgttctaattttctttctgttgtcttcattcttgtttatttttttaaataacttgctgtttaatatttttggctcaatttgatcaaaagtacgtgaatcacttcttgcagttacgtttcttacttgcgtttaaaacttactcgtgtggttcctttgggatataaaacatgctctgtggagtctgtctcccactgccgtctccaggcaagaaccacaatttaaagtagagattcttgtaaaggataaaggacctgtttatactggttttcttttaatcctgcacttagatgattgcagtattgttttagacaatttttatgtatttcatgccattctgggagttggtgttatagtttatttaattttgaattttgaagaattaaccagcagttaatccccagcctccttgcttctcagagtggattgtcaggtgacatcccttgaagtccctgaatggcttttgcctgggtcctcttccagatcgtccctactgcgtcatttggacttggttcatgtatccagacggcctcttttcaccatctagatccttgaagccaagaatgtagattatttgttgttttctttcttcagaatacttaacacatcactaggtgcagaaatgtgctcagcgcatgcatccactacctggaaaacctggcaaatgtgtgtctgtttcttttccagcaaagggaagcatcgtgctgaagtatgaaccagattctgcgtaagtcagtacacgccctgcccttagacacacacgccttgtcattagctacagtgttctcgttggtgaggaaagtgacttgggaaggtgggaaccttcttcaggccttttgtgattgttcctcagaagtagccttgtgaaaacttcagaaccaaaatattcctgtaggtcatcatgaagatgctgtttcatagaacactcagctcaggctaagtgaatttttacttacagagaataaaatgcatagtacttattttttaagcattagaaaccaatgaaaatttctgtcttgtacagagtaagtgtgaagattggacagttgagtgaggttccgttcttttgtcctcatggctggttcagccatgtgtgttgctgacatgttactgcatcaggagtgctagggccggagcatgggatttgctcacgcttgcactcacggtccccacgggaccactgctccacgctgcttgcatgtattcacgctgcatccttgtggggggccttgggggagcaggtgctggttttggcagcacttgccagaggaggaagccgggacagaaagttaggtggcttgcccagagctcctcagctcacgagtggcagaattcggatctcacctgcaacagctccgtcttggtgacatgcaacaccatttctgagaggaggaaacagaatggggtccacggctgactgggatttgggggagtgaataggtagaaccactgtacagatttctttgggaagttagtgttggaccagggatccagtgctaacttgaaggaaccaccaaagcgcagatcctgaggcaggtgcaagctgggggtcggggcgggcccagccaagtagggaaggaggagggataggtaagaggttggcagggaggccgaggggccacatgtcctcagggttcaggctgtccggctgccatgtggagagaggacaggtgggggctgaggtgggaggggcaggaacttggtgagggaacaacagatggatgggctggagaggtcagcgaggccgagcgcatgagcaccctggggacctgctggaggcggatggtgtctggatggtgaacgggcaggagcatctagtgattgatggcttctgggtgtttttaacgagagtttgaacaaagactcagaaatggtttttaaaataacagtcccatgtggcccacatagaaaatattgggatattttaaggtgtggattcacttttccatatttaaacacttgtttctacttggtgaaatacacaggtgacaggtcaacttcaggaataatggttttttttttttaagaagatgggagttgggaatttcttatattttcctctcacttcttaaaaccacctttgtgcccctgctttacattaggaaaaatggaaaggtgattaaacacggccgttaggagcctaaaatctaggtcagagtcccgtatgaaagaaatcagataagttgagagagggcgtgtgcaggttggaaatggtggcgtccatctctgctggggcgtcgatgcctcctggctggacaggtggagcctggaaggtagggaggctcggagcatgttcagagaggcagggatgtttttgactgtgggtaacactgcatgctgggaacggaaatgagcactgtgtgcccagcagggctcagactgccatgtggggtccctggagaagaaatcacgctaacactgagtatcaaaaggatgagtggtcctgggttctgctctggcagccctgctaagggcagagtctcccggtggtcctgggtcctgctctgacgccctgctaagggcagagtctcccagcctctcaggagcttcagtccacttaggaaacttgaggtgagctgcagggagcctgtggagaaacgcttctcatttagtggggtgaggtgacacggggtggcggggacagtaagttaagtttgagaaggaagtatgtagaaagtgtatgtgaaatctgtagatttggggcctctactccattgtgaacttaaagaggcagggcgaaagcgagggtgagaattttactcaggacatgaagaaggaagtcctttgagaaaacctacccctcggatccagatgaaatgtcctcccagtagttttgggggttttgtgggtatgggttttctgtggttttcttttttaagcagttgcatattacatctcattcgatttagttatgggaatgtttaatgttctacatgaagccactttcacttgtaggaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtgagcaggcccaaggcagcgggggcccacacccctcacacgcaaaactggcttcttctggtcactggtgtctgaaaccaaatccagagcagcctgtggcctgtaaagcatatatttctaatgactgcagactggtgggatcataggagccttctgaatgaccaggactgctttctttggagctgatgaaaatgtactcttttagcgtgttagaaatcacttgttttattttgtttctttggccaagctgggtctagtgtttcttttgctgggaatagactttcaaaagttgtacttctatcaagaaacaaaactgcccttgcagaaatttcaggtcttttgttaagcctgtattggtcttaaggtgcagtattttttaaattattatttatagaaagaatctataaattcttggggaagtgtgttataagctttaataattacattgagctgcacctcagtggtgtgtcattaacatgcagtggggttaatatctgaggcctcagatgactttgtgccttttggaataaagggtaaaataaactctcccagagtaagagctgtatcgtgaattgtcatactaattattgagggggacttatgtgcttttattgaatggagtgctttacaatttttatttttaaatggggttgggatccttggaatatttcaataaaattgataaaatata

SEQ ID NO: 4 - DNA, 2301 bp, Homo sapiens
atggggcggcggtcgcggggtcggcggctccagcaacagcagcggccggaggacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaagccacgcaaaagagattctccattgcttaaagaacaaatattttaaggaattggaggacctggaggtggacggtcagaaagttgaagttccacagccactgagttggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattgaggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacgggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccgg

SEQ ID NO: 5 - protein, 14 aa, Artificial
  antigen peptide for raising anti phosphorylated H3 histon antibody
ARKSTGGKAPRKQL

SEQ ID NO: 6 - protein, 10 aa, Artificial
  peptide fragment named as lep56
LFEHYYQELK

SEQ ID NO: 7 - protein, 10 aa, Artificial
  peptide fragment named as lep77-1
VPQPLSWYPE

SEQ ID NO: 8 - protein, 9 aa, Artificial
  peptide fragment named as lep77-2
LIEMLHADM

SEQ ID NO: 9 - protein, 10 aa, Artificial
  peptide fragment named as lep60
LESPSFTGTG

SEQ ID NO: 10 - DNA, 21 bp, Artificial
  primer for 5′-RACE
ctggtagtagtgctcgaacag

SEQ ID NO: 11 - DNA, 24 bp, Artificial
  primer for 5′-RACE
ataccaactcagtggctgtggaac

SEQ ID NO: 12 - protein, 11 aa, Artificial
  synthesized peptide corresponding to the C terminal region of SAK I
GCDPAGVHPPR

(19) United States
(12) Patent Application Publication
(10) Pub. No.: US 2009/0305255 A1
(43) Pub. Date: Dec. 10, 2009

(54) Substrate Protein for M-Phase Kinase and use Thereof

(75) Inventors: Masaaki Tatsuka, Hiroshima-shi (JP); Takashi Takata, Hiroshima-shi (JP)

Correspondence Address: BIRCH STEWART KOLASCH & BIRCH, PO BOX 747, FALLS CHURCH, VA, 22040-0747, US

(73) Assignees: HIROSHIMA UNIVERSITY, HIROSHIMA (JP); TWO CELLS CO., LTD., HIROSHIMA (JP)

(21) Appl. No.: 12/223,482

(22) PCT Filed: Feb. 1, 2006
(86) PCT No.: PCT/JP2006/302106

§ 371 Date: Mar. 17, 2009

(51) Int. Cl. C12Q 1/68; C12Q 1/48; G01N 33/53; C07K 16/18

(52) U.S. Cl. 435/6; 435/15; 435/7.1; 530/387.9

(57) ABSTRACT

An isolated protein of (a) a protein having an amino acid sequence of SEQ ID No. 2; (b) a protein having an amino acid sequence partially different from the amino acid sequence of SEQ ID No. 2 that is a substrate of M-period kinase and has nucleic acid-methylating activity suppressed by phosphorylation; (c) a protein having the amino acid sequence of SEQ ID No. 2 that is phosphorylated by M-period kinase; or (d) a protein having an amino acid sequence partially different from the amino acid sequence of SEQ ID No. 2 that is phosphorylated by M-period kinase.

### TECHNICAL FIELD

The present invention relates to an M-period kinase substrate protein (SAKI: Substrate of AIM-1 kinase) that is phosphorylated at the start of the mitotic period (M period) of cell cycle and dephosphorylated at the end of the M period and use of the same.

### BACKGROUND ART

AIM-1 (Aurora and Ipl1-like midbody-associated protein) gene is a gene found in animal cells as a resembling gene of mutant complementary genes associated with chromosome segregation, yeast Ipl1 and fly Aurora. Functional analysis of AIM-1 has been made for elucidation of the causes of chromosomal aneuploidy during carcinogenesis (Terada, Y., Tatsuka, M., Suzuki, F., Yasuda, Y., Fujita, S., and Otsu, M.: AIM1: a mammalian midbody-associated protein required for cytokinesis. EMBO J., 17: 667-676, 1998; and Tatsuka, M., Katayama, H., Ota, T., Tanaka, T., Odashima, S., Suzuki, F., and Terada, Y.: Multinuclearity and increased ploidy caused by overexpression of the aurora- and Ipl1-like midbody-associated protein mitotic kinase in human cancer cells. Cancer Res., 58: 4811-4816, 1998). On the other hand, it is known that, in addition to AIM-1, there are at least two kinds of analogous kinases (AIM-2 and AIM-3) in human (Katayama, H., Ota, T., Morita, K., Terada, Y., Suzuki, F., Katoh, O., and Tatsuka, M.: Human AIM-1: cDNA cloning and reduced expression during endomitosis in megakaryocyte-lineage cells. Gene, 224: 1-7, 1998). Currently, AIM-1 is classified as Aurora B, AIM-2 as Aurora A, and AIM-3 as Aurora C respectively.

AIM-1 is known to be an M-period passenger protein, based on analysis of its localization in animal cells during progress of the M period. Specifically, AIM-1 is localized in the chromosome centromere region in early M period and then, aligned together with the chromosome centromere on the equatorial plane when the chromosome is aligned on the equatorial plane. After segregation of the sister chromosomes, it is transported to the poles with sister chromosomes, but separates from the sister chromosome before they are pulled toward both poles, relocates itself in the central region of the M-period cell and engages in forming a structure for contraction of cytoplasm (Murata-Hori, M., Tatsuka, M, and Wang, Y. L.: Probing the Dynamics and Functions of Aurora B Kinase in Living Cells during Mitosis and Cytokinesis., Mol. Biol. Cell. 13: 1099-1108, 2002). On the other hand, AIM-1 is a M-period passenger protein during progress of the M period, and functions together with survivin, which is known to inhibit M-period apoptosis, and an inner centromere protein (INCEP), which is known as a centromere protein, by forming complexes with them (Temme, A., Rieger, M., Reber, F., Lindemann, D., Weigle, B., Diestelktter-Bachert, P., Ehninger, G., Tatsuka, M., Terada, Y., and Rieber, E. P.: Localization, Dynamics and Function of Survivin Revealed by Expression of Functional SurvivinDsRed Fusion Proteins in the Living Cell. Mol. Biol. Cell 14: 78-79, 2003). AIM-1 also functions as an important signal molecule for initiation of contraction for cytoplasmic separation after segregation of sister chromosomes, (Terada, Y., Tatsuka, M., Suzuki, F., Yasuda, Y., Fujita, S., and Otsu, M.: AIM1: a mammalian midbody-associated protein required for cytokinesis. EMBO J., 17: 667-676, 1998; Tatsuka, M., Katayama, H., Ota, T., Tanaka, T., Odashima, S., Suzuki, F., and Terada, Y.: Multinuclearity and increased ploidy caused by overexpression of the aurora- and Ipl1-like midbody-associated protein mitotic kinase in human cancer cells. Cancer Res., 58: 4811-4816, 1998; Katayama, H., Ota, T., Morita, K., Terada, Y., Suzuki, F., Katoh, O., and Tatsuka, M.: Human AIM-1: cDNA cloning and reduced expression during endomitosis in megakaryocyte-lineage cells. Gene, 224: 1-7, 1998; and Kawasaki, A., Matsumura I., Miyagawa, J., Tanaka, H., Terada, Y., Tatsuka, M., Machii, T., Furukawa, Y., and Kanakura Y.: Down-regulation of an AIM-1 kinase couples with megakaryocytic endomitosis of human hematopoietic cells. J. Cell Biol. 152: 257-287, 2001), and is involved in cytoplasmic separation tightly by phosphorylating intermediate filaments such as myosin, desmin and vimentin, MgcRacGAP, and the like (Murata-Hori, M., Tatsuka, M, and Wang, Y. L.: Probing the Dynamics and Functions of Aurora B Kinase in Living Cells during Mitosis and Cytokinesis. Mol. Biol. Cell. 13: 1099-1108, 2002; Goto, H., Yasui, Y., Kawajiri, A., Nigg, E. A., Terada, Y., Tatsuka, M., Nagata, K., and Inagaki, M.: Aurora-B regulates the cleavage furrow-specific vimentin phosphorylation in the cytokinetic process. J. Biol. Chem. 278: 8526-8530, 2003; Kawajiri, A., Yasui, Y., Goto, H., Tatsuka, M., Takahashi, M., Nagata, K., and Inagaki, M.: Functional Significance of the Specific Sites Phosphorylated in Desmin at Cleavage Furrow: Aurora-B May Phosphorylate and Regulate Type III Intermediate Filaments during Cytokinesis Coordinatedly with Rho-kinase. Mol. Biol. Cell 14: 1489-1500, 2003; and Minoshima, Y., Kawashima, T., Hirose, K., Tonozuka, Y., Kawajiri, A., Bao, Y. C., Deng, X., Tatsuka, M., Narumiya, S., May, W. S., Nosaka, T., Semba, K., Inoue, T., Satoh, T., Inagaki, M., and Kitamura, T.: Phosphorylation by Aurora B Converts MgcRacGAP to a RhoGAP during Cytokinesis. Dev. Cell 4: 549-560, 2003).

On the other hand, AIM-1 and also the other two kinds of Aurora kinases are expressed abundantly in cancer cells. The high expression of these kinases seems to be closely related to the abnormality-generating mechanism in chromosome segregation process during progress of carcinogenesis (Tatsuka, M., Katayama, H., Ota, T., Tanaka, T., Odashima, S., Suzuki, F., and Terada, Y.: Multinuclearity and increased ploidy caused by overexpression of the aurora- and Ipl1-like midbody-associated protein mitotic kinase in human cancer cells. Cancer Res., 58: 4811-4816, 1998; Katayama, H., Ota, T., Jisaki, F., Ueda, Y., Tanaka, T., Odashima, S., Suzuki, F., Terada, Y. and Tatsuka, M.: Mitotic kinase expression and colorectal cancer progression. J. Natl. Cancer Inst. 91: 1160-1162, 1999; Katayama H., Zhou H., Li Q., Tatsuka M., and Sen S.: Interaction and feedback regulation between STK15/BTAK/Aurora-A kinase and protein phosphatase 1 through mitotic cell division cycle. J. Biol. Chem. 276: 46219-46224, 2001; and Ota, T., Suto, S., Katayama, H., Han, Z-B., Suzuki, F., Maeda, M., Tanino, M., Terada, Y., and Tatsuka M.: Increased mitotic phosphorylation of histone H3 due to AIM-1/Aurora-B overexpression contributes to chromosome number instability. Cancer Res. 62: 5168-5177, 2002).

See JP-A-11-164694 (“JP-A” means unexamined published Japanese patent application) for other information on AIM-1.

### DISCLOSURE OF INVENTION

A task of the present invention is to provide a functionally identified protein that can be a substrate for M-period kinases such as AIM-1, which are active in the M period. Another task of the present invention is to understand the function of the substrate in the body and facilitate use thereof.

The inventors have made intensive studies to achieve the tasks above. Specifically, the inventors have made studies, aimed at identifying the substrate for AIM-1 and elucidating the function or the role thereof in the body. Possible application of the substrate in the fields of research and medicine was also studied.

First, the inventors tried to identify a H3 histone-phosphorylating enzyme in cell. As a result, AIM-1 was shown to be the M-period phosphorylating enzyme. On the other hand, in addition to H3 histone, there was identified a substrate protein that was phosphorylated by AIM-1. The inventors paid attention to the substrate protein and tried to identify the protein. As a result, a cDNA coding the protein (SEQ ID No. 1) was cloned successfully. A protein of estimated 767 amino acid residues (SEQ ID No. 2) was found to be coded in the ORF (open reading frame) region of the obtained cDNA. The protein was designated as SAKI.

Further studies revealed SAKI's site phosphorylated by the kinase and gave beneficial information about distribution of SAKI expression and localization in cell. Alternatively, SAKI's overexpression was found to lead to increase in incorporation of methyl groups into rRNA, suggesting that SAKI had an activity to methylate rRNA in cell. More specifically, SAKI was considered to be a methylating enzyme associated with the maturation process of rRNA. In-vitro nucleic acid-methylating assays of SAKI showed that DNA was not used as a substrate, suggesting that SAKI was an RNA-methylating enzyme having no DNA-methylating enzyme activity. Further, it was found that SAKI was converted into an inactive form by phosphorylation by kinase (phosphorylated SAKI) and thus, the methylation activity was inhibited.

Subsequent intensive studies on application of SAKI in the medical field showed that high expression of SAKI was observed in all cases of various colon cancer cell lines and oral cavity squamous cell carcinoma cell lines. In addition, amplification of the SAKI gene was observed in HeLa cell cultures in which SAKI is expressed abundantly. Amplification of the SAKI gene was observed also in clinical autopsy tissues. On the other hand, immunostaining of human cancer tissues by using an anti-SAKI antibody showed dyeing affinity more favorable than that by using conventional antibodies. Thus, expression of SAKI in various cancer tissues was studied, to show that high expression of SAKI was observed in all cases studied. These results demonstrate that SAKI is useful as an indicator for use in diagnosis of human cancer. In addition, results of immunoblotting by using a SAKI antibody revealed that it was possible to detect a single cancer cell in a sample containing 100 to 1000 cells, indicating that the SAKI detection was quite useful for identification of cancer cell and increase in activity caused by SAKI's overexpression is tightly related to expression of carcinogenesis. It was considered collectively based on these findings, that it was possible to control the activity of SAKI, by suppressing SAKI's overexpression and by converting activated dephosphorylated SAKI into the phosphorylation state, and also that such suppression or inhibition of SAKI activity was effective for preventing canceration of cell and progress of cancer.

The present invention, which was made based on the findings above, has the following aspects.

An aspect of the present invention is an isolated protein, selected from the group consisting of the following proteins (a), (b), (c) and (d): (a) the protein having the amino acid sequence of SEQ ID No. 2; (b) a protein having a sequence partially identical with the amino acid sequence of SEQ ID No. 2 that is a substrate of M-period kinase and has nucleic acid-methylating activity suppressed by phosphorylation; (c) the protein having the amino acid sequence of SEQ ID No. 2 that is phosphorylated by M-period kinase; and (d) a protein having a sequence partially different from the amino acid sequence of SEQ ID No. 2 that is phosphorylated by M-period kinase.

Another aspect of the present invention is an isolated nucleic acid, selected from the group consisting of the following nucleic acids (A), (B) and (C): (A) the nucleic acid coding the protein according to the above (a), (b), (c) and (d); (B) a nucleic acid having a nucleotide sequence complementary to the nucleotide sequence coding the protein according to the above (a), (b), (c) and (d); and (C) a nucleic acid hybridizing with the complementary chain of the nucleic acid (A) or (B) under stringent condition.

Yet another aspect of the present invention is a vector including the nucleic acid according to the present invention, preferably in the form of expression vector.

Yet another aspect of the present invention is a cell containing the nucleic acid according to the present invention externally incorporated. Typically, the cell is constructed while the vector is incorporated into a host cell.

Yet another aspect of the present invention is a method of producing a protein functioning as a substrate of M-period kinase, comprising the following steps (a1) and (b1): (a1) a step of culturing the cell according to the present invention under a condition suitable for production of the protein coded by the nucleic acid; and (b1) a step of recovering the produced protein.

The present invention also provides an antibody to the protein according to the present invention (protein having the amino acid sequence of SEQ ID No. 2 or the homologous protein thereof).

Yet another aspect of the present invention relates to a method of determining the malignancy of an analyte cell (malignancy-determining method). The malignancy-determining method according to the present invention includes a step of determining the amount of the protein according to the present invention in an analyte cell isolated from the body. The protein amount is preferably determined by using an immunological staining method. The present invention also provides a method of determining the malignancy of an analyte cell, comprising a step of determining the amount of the nucleic acid according to present invention present in an analyte cell isolated from the body, instead of the step described above.

The present invention also provides a reagent for use in the method above. The reagent contains an antibody specifically binding to the protein according to the present invention or a nucleic acid hybridizing with the complementary chain of the nucleic acid according to the present invention under stringent condition. The present invention also provides a kit containing the reagent (kit for determination of the malignancy of an analyte cell). The kit includes an instruction manual.

Yet another embodiment of the present invention relates to a method of screening an compound effective to a disease characterized by abnormality in expression amount of the protein of the present invention or in the content of the nucleic acid of the present invention, the method comprising a step of examining the presence and the degree of an analyte compound inhibiting the binding between the protein according to the present invention and the M-period kinase. In yet another embodiment, provided is a screening method including a step of examining the presence and the degree of an analyte compound inhibiting the nucleic acid-methylating activity of the protein according to the present invention, replacing the step above.

As will be described in detail in Examples, the M-period kinase substrate protein (SAKI) newly identified by the inventors is phosphorylated in the M period and exerts nucleic acid-methylating activity in the dephosphorylated state normally in the interphase. It is involved in the maturation process of rRNA with the nucleic acid-methylating activity. The SAKI is phosphorylated specifically in the M period, losing SAKI's methylating activity. These facts indicate that SAKI or the nucleic acid coding the same, for example, is useful in screening therapeutic or diagnostic drugs to diseases or syndromes associated with activation of rRNA maturation and methylation or in developing a treatment or diagnosis method. The amount of the nucleic acid coding SAKI and SAKI is overexpressed in various cancer cells, indicating that SAKI or the nucleic acid coding the same is useful in determining cellular malignancy as detection object (indicator or marker).

In the present specification, the term “comprising” or “including” is used as an expressing including “consisting of”.

Other and further features and advantages of the invention will appear more fully from the following description, appropriately referring to the accompanying drawings.

### BRIEF DESCRIPTION OF DRAWINGS

FIG. 1 shows the results obtained when a FLAG-tagged wild AIM-1-expressing vector, an Aik-expressing vector, and a kinase-deletion-mutant protein-expressing vector wherein the ATP-binding site lysine is substituted with alanine were introduced into HeLa cell, and the cell was immunized with an anti-FLAG antibody, DNA staining, and an anti-H3 histone Ser-10-phosphorylated site antibody. The results show that AIM-1 functions as a H3 histone-phosphorylating enzyme in animal cell.

FIG. 2 shows the results of immunoblotting by using an antibody recognizing the serine site phosphorylated by AIM-1. empty: pcDNA3, WT: pcDNA3 FLAG-AZM-1, K/R: pcDNA3 FLAG-AIM-1-K/R, exp: logarithmic-proliferation-period cell, Noc: M-period cell with Nocodazole treatment for 18 hours (described also in EMBJ. 17, 667, 1998 for examples of WT and K/R). The 15 kDa phosphorylated H3 histone band is suppressed by expression of AIM-1-K/R (AIM-1 kinase-deleted mutant protein). Simultaneously, expression of the 100 kDa protein is also suppressed.

FIG. 3 shows the results obtained when HeLa cell is synchronized by double-thymidine method, the released cells were sampled over time and analyzed by immunoblotting by using an antibody recognizing the serine site phosphorylated by AIM-1. Lanes from left to right correspond respectively to samples of post-synchronization culture for 6 hours, 7 hours, 8 hours, 9 hours, 10 hours, 11 hours, 12 hours, 13 hours, and 14 hours. The 100 kDa phosphorylated band appears almost simultaneously with the 15 kDa histone H3 phosphorylated band.

FIG. 4 shows the results of immunoprecipitation experiment by using the cell released from the HeLa cells after synchronization (6 and 10 hours after release). The left column shows the results after electrophoresis of the immunoprecipitation product and subsequent CBB staining. There is a 100 kDa band 10 hours after release, which was not observed 6 hours after release. The right column shows the results obtained when the gel after electrophoresis was analyzed by immunoblotting by using an antibody recognizing the serine site phosphorylated by AIM-1. Sup represents culture supernatant, while I.P. represents the immunoprecipitation product.

FIG. 5-1 is a chart showing the SAKI full-length cDNA and the amino acid sequence in the estimated coding region. The amino acid shown in italic characters in the amino acid sequence is a peptide sequence identified by amino acid sequence analysis. The underlined region in the nucleotide sequence represents a primer region used in analysis by 5′-RACE method.

FIG. 5-2 is also a chart showing the SAKI full-length cDNA and the amino acid sequence in the estimated coding region (continued from FIG. 5-1). The amino acid sequence shown in italic characters in the amino acid sequence above is a peptide sequence identified by amino acid sequence analysis.

FIG. 5-3 is also a chart showing the SAKI full-length cDNA and the amino acid sequence in the estimated coding region (continued from FIG. 5-2). The amino acid sequence shown in italic characters in the amino acid sequence above is a peptide sequence identified by amino acid sequence analysis.

FIG. 5-4 is also a chart showing the SAKI full-length cDNA and the amino acid sequence in the estimated coding region (continued from FIG. 5-3).

FIG. 6 shows the results obtained when a mutant gene (SAKI-SA) wherein the SAKI's 139th serine residue is substituted with alanine is expressed in HeLa cell, treated with Nocodazole, and then, analyzed by immunoblotting by using an antibody recognizing the serine site phosphorylated by AIM-1. The results show that substitution of the 139th serine suppresses phosphorylation of SAKI.

FIG. 7 shows an expression pattern of the SAKI protein when a HeLa cell synchronization system is used. The sample of the synchronized cell is the same as that used in FIG. 3. The results were obtained by using an anti-C-terminal SAKI antibody, but similar results were obtained when an anti-full-length SAKI antibody was used.

FIG. 8 shows the results of immunoprecipitation experiment of samples obtained from synchronized HeLa cells (6 hours: interphase, 10 hours: M period). The left figure shows the results obtained when the cell is immunoprecipitated with an anti-C-terminal SAKI antibody and analyzed by immunoblotting by using an anti-phosphorylated Ser10 antibody; the central figure shows the results obtained when the cell is immunoprecipitated with an anti-phosphorylated Ser10 antibody and analyzed by immunoblotting with an anti-C-terminal SAKI antibody; and the right figure shows the results obtained when the cell is immunoprecipitated with an anti-C-terminal SAKI antibody and analyzed by immunoblotting by using an anti-C-terminal SAKI antibody.

FIG. 9 shows intracellular localization of the SAKI protein. NHDF cell was fixed with methanol and then, immunostained with an anti-SAKI antibody. C23 is known as a nucleolus protein. In addition, multiplex staining of B2 trinuclear corpuscle protein and SAKI also confirmed localization in nucleolus in the SAKI interphase cell (data not shown).

FIG. 10 shows acceleration effect of rRNA methylation by forced expression of SAKI in HeLa cell. Lane 1 represents only experimental operation (MOCK); lane 2 shows results obtained with an empty vector; and lane 3 shows results obtained with a vector containing pcDNA3.1-SAKI introduced.

FIG. 11 shows the results obtained by analyzing SAKI DNA-methylating enzyme activity. 1 μg of λDNA was after-treated with a DNA-methylating enzyme (Sss1 methylase, human hemimethylase Dnmt1) and a SAKI protein (MOCK: SAKI-free, 6 hr: synchronized interphase HeLa cell SAKI immunosediment, 10 hr: synchronized M-period HeLa cell SAKI immunosediment, non: E. coli-producing His-SAKI protein only, GST-AIM-1-WT: sample of E. coli-producing His-SAKI protein phosphorylated in vitro with E. coli-producing active-form GST-AIM-1-WT, AIM-1KR: sample of E. coli-producing His-SAKI protein phosphorylated in vitro with E. coli-producing kinase-activity-deficient-form GST-AIM-1-KR, Sss1: E. coli CpG methylase, or Dnmt1: human hemimethylase), and the product was digested with 10 units of a restriction enzyme BstU1 for 1 hour, before electrophoresis in agarose-gel. There is a BstU1 noncleavage site (DNA-methylating activity) only in E. coli methylase Sss1. The results suggested that SAKI did not have the methyl group-transferring enzyme activity both on the strands of a double-stranded DNA.

FIG. 12 shows the results obtained by analyzing SAKI's DNA-methylating enzyme activity. The DNA-methylating enzyme activity was measured, as Poly-dI:dC was used as the substrate. Lane 1: MOCK, lane 2: Dnmt1, lane 3: GST-AIM-1-WT, lane 4: GST-AIM-1-KR, lane 5: GST-AIM-1-WT+His-SAKI-WT (0.1 μg)+kinase reaction, lane 6: GST-AIM-1-WT+His-SAKI-WT (0.5 μg)+kinase reaction, lane 7: GST-AIM-1-WT+His-SAKI-WT (1.0 μg)+kinase reaction, lane 8: GST-AIM-1-WT+His-SAKI-WT (2.0 μg)+kinase reaction. GST-AIM-1-WT had a DNA-methylating activity similar to that of Dnmt1. However, His-SAKI had no DNA-methylating activity. Because GST-AIM-1-KR had no DNA-methylating activity, SAKI's DNA-methylating activity seems to be expressed together with the kinase activity of AIM-1.

FIG. 13 shows the results obtained by analyzing SAKI's rRNA methylase activity. As shown in FIG. 10, SAKI accelerates rRNA methylation, but the 139th phosphorylated SAKI does not have such methylating enzyme activity. On the other hand, nonphosphorylated SAKI has a methylating enzyme activity less suppressed even in the M period. Thus, the activity of SAKI is controlled negatively by phosphorylation of the 139th serine.

FIG. 14 shows the results obtained by immunoblotting by using an antibody recognizing the serine site phosphorylated by AIM-1. SAKI is phosphorylated by aurora kinase (AIM-1) in the cell-cycle M period, but there was no SAKI phosphorylation when the aurora kinase was inhibited. The result confirmed that SAKI was a substrate of AIM-1.

FIG. 15 shows expression patterns of SAKI in various human internal organs. The SAKI protein was extracted from each organ and immunoblotted by using an anti-full-length SAKI antibody. The immunoblotting with the anti-C-terminal SAKI antibody is lower in sensitivity and thus, proteins are detected less easily in normal tissues.

FIG. 16 is a chart comparing expression of SAKI between in HeLa cell and in normal human fibroblast. Expression in testis was used for electrophoresis as a control for relative comparison with the results in FIG. 15. Immunoblotting was performed by using an anti-C-terminal SAKI antibody.

FIG. 17 shows the results obtained by analyzing SAKI gene by using genomic DNA by Southern blotting method. Full-length SAKI cDNA was used as the probe. Gene amplification was observed in HeLa cells and oral cancer cell lines and in oral cancer patient tissues. Lanes 1 to 6: oral cancer cell lines, lanes 7 to 20: oral cancer patient tissues, lane 21: NHDF, and lane 22: HeLa cell

FIG. 18 shows SAKI's expression states (immunostaining images) in various cancer tissues. ABC color development was performed by using an anti-SAKI C-terminal antibody. High expression of SAKI was observed in the tissues of skin cancer, oral cancer, breast cancer, and renal cancer.

FIG. 19 is a table summarizing the immunohistological search results of SAKI by using various human cancer tissues.

FIG. 20 includes chromatic images of SAKI in prostate gland tumor. The SAKI staining image (left) was compared with its corresponding hematoxylin staining image (right). The region identified as prostate cancer lesion by hematoxylin staining is stained distinctively by SAKI.

FIG. 21 shows the results of immunohistologic staining by using an anti-C-terminal SAKI antibody. FIG. 21(a) is an image of oral cancer tissue; and FIG. 21(b) shows the results obtained by analyzing the difference in dye affinity between the oral cancer tissue and the neighboring normal tissue.

FIG. 22 shows the result of immunohistologic staining by, using an anti-C-terminal SAKI antibody. FIG. 22(a) is an image showing a lung cancer tissue, and FIG. 22(b) shows the results obtained by analyzing the difference in dye affinity between the lung cancer tissue and the neighboring normal tissue.

FIG. 23 shows the results of comparison between the expression state of SAKI and that of Ki-67 commonly used as a typical pathological diagnostic growth marker by immunohistologic staining. (a) Ki-67 was scatteredly expressed only in cells in proliferation period, while (b) SAKI was expressed in almost all cancer cells.

FIG. 24 shows the results obtained when various numbers of HeLa cells were mixed with mouse BALB/c 3T3 A31-1-1 cells (10⁵ cells) and the cell lysate was analyzed by Western blotting (immunoblotting). The results showed that the SAKI detection limit was 100 to 1000 cancer cells or more in 10⁵ A31-1-1 cells.

FIG. 25 shows the results obtained when proteins extracted from the intracelial swab (patient sample) of a uterus cancer patient were analyzed by an immunoblotting method by using an anti-C-terminal SAKI antibody. As shown in this Figure, there was a positive reaction to the anti-C-terminal SAKI antibody in the region of around 100 kDa observed only in the sample of the grade-5 patient (lane 1).

### BEST MODE FOR CARRYING OUT THE INVENTION

Hereinafter, the specific modes of the present invention will be described in detail.

An aspect of the present invention relates to a newly identified protein that is phosphorylated by a M-period kinase and has a nucleic acid-methylating activity that is suppressed by phosphorylation. Thus, it relates to an isolated M-period kinase substrate protein (SAKI) and its biologically active region. It also relates to SAKI phosphorylated by the M-period kinase and its biological region. Hereinafter, the protein and the regions are also called collectively as “SAKI and the like” or “the protein and the like according to the present invention”.

The protein or the like according to the present invention can be isolated and prepared from natural materials, when it is a natural material, for example from cells and organisms containing the same by a standard method. The protein or the like according to the present invention may be produced by a recombinant DNA technique. Alternatively, the protein or the like according to the present invention may be produced by chemical synthesis. Many peptide synthesizers are commercially available, and one of them may be chosen and used for production of the protein or the like according to the present invention.

If the protein or the like according to the present invention is a natural material-derived product, the term “isolated”, as used in relevance to the protein or the like according to the present invention means a state that the protein contains substantially no component other than the desired protein in the natural material (in particular, substantially no contaminant proteins). In such a case, the phrase “contains substantially no contaminant proteins” means that the content of the contaminant proteins in the isolated protein or the like according to the present invention is, for example less than 20%, preferably less than 10%, and more preferably less than 5%, and still more preferably less than 1% by weight in the entire protein.

On the other hand, if the protein or the like according to the present invention is a product produced by a recombinant DNA technique, the term “isolated” means a state of the desired protein substantially containing no other components derived from the host cell, culture solution, or the like. In such a case, the phrase “containing substantially no contaminant protein” means that the content of the contaminant component is, for example less than 20%, preferably less than 10%, more preferably less than 5%, and still more preferably less than 1% by weight in the isolated protein or the like according to the present invention.

Alternatively, if the protein or the like according to the present invention is a protein produced by chemical synthesis, the term “isolated” means a state that the protein contains substantially no precursors (raw materials) or chemicals or the like used in the synthetic process. In such a case, the phrase “containing substantially no contaminant protein” means that the content of the precursor or the like is for example less than 20%, preferably less than 10%, more preferably less than 5%, and still more preferably less than 1%, by weight in the isolated protein or the like according to the present invention.

In the present description, the term “isolated” is the same as the term “purified”.

In the present description, the “biologically active region” is a region associated with the nucleic acid-methylating activity in SAKI or the like according to the present invention. Therefore, it is a region where modification (deletion, substitution, insertion, addition, or others of amino acids) thereof leads to elimination or deterioration of the nucleic acid-methylating activity. As will be described below in Examples, it was shown that the methylating activity of SAKI was exhibited by phosphorylation of a specific amino acid (139th serine). Thus, the amino acid region was found to be important in the SAKI's methylating activity. Therefore, a typical example of the biologically active region is a continuous region containing the amino acid region.

The biologically active region can be prepared by partial decomposition of the protein according to the present invention, or by a recombinant DNA technique.

An embodiment of the protein or the like according to the present invention has an amino acid sequence of SEQ ID No. 2. Alternatively in another embodiment, the present invention provides a protein having the same function as the protein above but being different in the amino acid sequence. In yet another embodiment, the present invention provides the protein having the amino acid sequence of SEQ ID No. 2 that is phosphorylated by the M-period kinase and a protein having the same function as that of the protein that is different in its partial amino acid sequence and phosphorylated by the M-period kinase. Hereinafter, these proteins will be called “homologous proteins”.

The phrase “different in partial amino acid sequence” specifically means alteration (change) in amino acid sequence caused by deletion or substitution of one or more amino acids, addition or insertion of one or more amino acids, or the combination thereof which constitute the amino acid sequence. The difference in amino acid sequence is permitted in the range that the protein can be a substrate of the M-period kinase and the nucleic acid-methylating activity is retained. The site of amino acid sequence difference is not particularly limited, and there may be alteration at multiple sites, if the requirements above are satisfied. The “multiple” in the multiple site is, for example, is a number less than 30% of the total number of the amino acids, and is preferably a number of less than 20%, more preferably less than 10%, still more preferably less than 5%, and most preferably less than 1%. Thus, the homologous protein has a homology for example of 70% or more, preferably 80% or more, more preferably 90% or more, still more preferably 95% or more, and most preferably 99% or more with the amino acid sequence of SEQ ID No. 2.

Preferably, the homologous protein is prepared by causing conservative amino acid substitution at nonessential amino acid residues (amino acid residues not involved in being a substrate protein for the M-period kinase and its nucleic acid-methylating activity). The “conservative amino acid substitution” is substitution of an amino acid residue with an amino acid residue having a similar side-chain. Amino acid residues are grouped, depending on the side chain, into several families: a basic side-chain amino acid family (e.g., lysine, arginine, histidine), an acidic side-chain amino acid family (e.g., asparaginic acid, glutamic acid), a non-changed polar side-chain amino acid family (e.g., glycine, asparagine, glutamine, serine, threonine, tyrosine, cysteine), a non-polar side-chain amino acid family (e.g., alanine, valine, leucine, isoleucine, proline, phenyl alanine, methionine, tryptophan), a β-branched side-chain amino acid family (e.g., threonine, valine, isoleucine), and an aromatic side-chain amino acid family (e.g., tyrosine, phenyl alanine, tryptophan, histidine). The conservative amino acid substitution is a substitution between amino acid residues in the same family.

The homology (%) between two amino acid or nucleic acid sequences (hereinafter, referred to as “two sequences,” including both amino acid and nucleic acid sequences) is determined, for example, by the following procedure. First, two sequences are aligned properly for optimal comparison (e.g., the first sequence may be expressed with an inserted gap for optimal alignment with the second sequence). If the molecule at a specific position in the first sequence (amino acid residue or nucleotide) is the same as that at the corresponding position of the second molecule, the molecules in these positions are the same as each other. The homology between two sequences is a function of the number of the positions homologous in the two sequences (i.e., homology (%)=number of homologous positions/total number of positions×100), and preferably, the number and the size of gaps needed for optimized alignment are also taken into consideration.

Comparison between two sequences and determination of homology can be done, based on a mathematical algorithm. Typical examples of the mathematical algorithms for comparison of sequences include that described in Karlin and Altschul (1990) Proc. Natl. Acad. Sci. USA 87:2264-68 and that described in Karlin and Altschul (1993) Proc. Natl. Acad. Sci. USA 90:5873-77, but are not limited thereto. Such an algorithm is incorporated in the NBLAST program and the XBLAST program (version 2.0) described in Altschul et al., (1990) J. Mol. Biol. 215: 403-10. To obtain a nucleotide molecule sequence homologous to the nucleic acid molecule according to the present invention, the BLAST nucleotide retrieval is conducted with NBLAST program, for example, at a score of 100 and a word length of 12. To obtain an amino acid sequence homologous to the polypeptide molecule according to the present invention, the BLAST polypeptide retrieve is performed according to the XBLAST program, for example, at a score of 50 and a word length of 3. To obtain a gap alignment for comparison, the Gapped BLAST described in Altschul et al., (1997) Amino Acids Research 25 (17):3389-3402 may be used. In using the BLAST and Gapped BLAST, default parameters of the corresponding programs (e.g., XBLAST and NBLAST) can be used. See http://www.ncbi.nlm.nih.gov for details. An example of other usable mathematical algorithm for comparison of sequences is the algorithm described in Myers and Miller (1988) Comput. Appl. Biosci. 4: 11-17. Such an algorithm is incorporated, for example, in the ALIGN program for use in GENESTREAM network servers (IGH Montpellier, France) or ISREC servers. When the ALIGN program is used for comparison of amino acid sequences, the calculation can be carried out, for example, by using PAM120 residue mass table at a gap length penalty of 12 and a gap penalty of 4.

The homology between two amino acid sequences can be determined by using the GAP program in GCG software package and Blossom 62 or PAM 250 matrix at a gap weight of 12, 10, 8, 6, or 4 and at a gap length weight of 2, 3, or 4. The homology between two nucleic acid sequences can also be determined by using the GAP program GCG in software package (http://www.gcg.com) at a gap weight of 50 and a gap length weight of 3.

In yet another embodiment, the present invention provides a fusion protein formed by binding of the protein or the like according to the present invention to another molecule. The other molecule is, for example, a polypeptide (including signal peptide) or a labelling substance (such as GST). It is possible to strengthen or assist the function of the polypeptide or the like according to the present invention, add other functions, and accelerate expression and secretion thereof during recombinant production (in the case of signal peptide) and facilitation of purification, by bonding of the protein with such an additional molecule.

The fusion protein can be prepared by a standard recombinant DNA technique. For example, first, a DNA fragment coding the protein or the like according to the present invention and a DNA fragment coding another molecule are prepared and in-frame bonded to each other. The DNA coding the fusion protein thus obtained is expressed in a suitable cell, and the product is isolated and purified by a standard biochemical technique.

SAKI or the like or part thereof may be used as an immunogen for obtaining an antibody to the SAKI or the like (hereinafter, referred to as “anti-SAKI antibody”). Thus, the present invention provides a protein or a peptide (immunogen) that can induce the anti-SAKI antibody.

Another aspect of the present invention relates to an antibody (anti-SAKI antibody) specifically binding to SAKI or the homologous proteins (SAKI or the like). The term “antibody” is a concept including polyclonal antibodies, monoclonal antibodies, chimera antibodies, single-stranded chain antibodies, CDR graft antibodies, humanized antibodies, and the fragments thereof (however, capable of specifically binding to SAKI or the like). The antibody according to the present invention can be prepared, for example, by immunological method, phage display method, or ribosome display method.

Production of a polyclonal antibody by immunological method is carried out in the following procedure. An antigen (SAKI or the like) is prepared, and an animal such as mouse, rat, rabbit, or goat is immunized. SAKI or the homologous protein, or part thereof may be used as the antigen. If an antigen is less immunogenic because of low-molecular weight, a carrier protein-bonded antigen is used favorably. Examples of the carrier proteins for use include KLM (Keyhole Light Hemocyanin), BSA (Bovine Serum Albumin), OVA (Ovalbumin) and the like. Alternatively, carbodiimide method, glutaric aldehyde method, diazo condensation method, or MBS (maleimide benzoyloxyimide succinate) method may be used, for example, for binding of the carrier protein. Immunization is repeated as needed, and the blood is collected when the antibody titer becomes sufficiently high, and the blood serum is obtained for example by centrifugation. The antiserum thus obtained is purified by affinity chromatography, to give a polyclonal antibody.

Alternatively, a monoclonal antibody can be prepared in the following procedure. First, an animal is immunized in a similar manner to the procedure above. Immunization is repeated as needed, and antibody production cells are collected from the immunized animal when the antibody titer becomes sufficiently high. Then, the antibody production cells obtained are hybridized with a myeloma cell. The hybridoma is then made monoclonal, and a clone producing an antibody showing high specificity to the desired protein is selected. Purification of the culture solution of the selected clone gives a desired antibody. Alternatively, the hybridoma may be grown to a desirable number or more and then injected into an animal (e.g., mouse) intraperitoneally, and then, the ascites containing grown hybridoma may be collected and purified for preparation of the desired antibody. Affinity chromatography for example by using protein G or protein A is used favorably for purification the culture solution or the ascites. Alternatively, antigen-immobilized affinity chromatography may be used instead. Yet alternatively, methods such as ion exchange chromatography, gel filtration chromatography, ammonium sulfate fractionation, and centrifugation can also be used. These methods may be used alone or in any combination.

As for the process for producing the antibody, see also Kohler and Milstein (1975) Nature 256: 495-497; Brown et al. (1981) J. Immunol. 127: 539-46; Brown et al. (1980) J. Biol. Chem. 255: 4980-83; Yeh et al. (1976) Proc. Natl. Acad. Sci. USA 76: 2927-31; Yeh et al. (1982) Int. J. Cancer 29: 269-75; Kozbor et al. (1983) Immunol. Today 4: 72; Kenneth, R. H. in Monoclonal Antibodies: A New Dimension in Biological Analyses, Plenum Publishing Corp., New York, N.Y. (1980); Lerner, E. A. (1981) Yale J. Biol. Med. 54: 387-402; Gefter, M. L. et al. (1977) Somatic Cell Genet. 3: 231-36, and others.

As for the phage display method, see various literatures, for example including Huse et al. (1989) Science 246: 1275-1281; McCafferty et al. (1990) Nature 348: 552-554; Fuchs et al. (1991) Bio/Technology 9: 1370-1372; Barbas et al. (1991) Proc. Natl. Acad. Sci. USA 88: 7978-7982; Hoogenboom et al. (1991) Nucleic Acids Res. 19: 4133-4137; Gram et al. (1992) Proc. Natl. Acad. Sci. USA 89: 3576-3580; Hay et al. (1992) Hum. Antibod. Hybridomas 3: 81-85; Griffiths et al. (1993) EMBO J. 12: 725-734; PCT International Publication WO90/02809; PCT International Publication WO92/20791; PCT International Publication WO92/15679; PCT International Publication WO92/09690; and others. Kits for preparation and screening of phage display libraries are commercially available, and one of them may be used favorably.

In an embodiment of the present invention, it provides an antibody specifically binding to the SAKI phosphorylated by M-period kinase (i.e., SAKI with the 139th serine phosphorylated). Use of the antibody, for example, allows selective detection of the phosphorylated SAKI and evaluation of the degree of activation by M-period kinase (in other words, amount of activated M-period kinase) and the degree of suppression of SAKI activity (in other words, amount of activated SAKI). The information thus obtained is useful for evaluation and estimation of the phenomena associated with M-period kinase or SAKI. For example, it is useful in diagnosis of cancers, as will be described below in Examples. The antibody specifically binding to the phosphorylated SAKI can be prepared, for example, by immunizing an animal with a SAKI's partial peptide including the 139th phosphorylated serine residue as antigen in the procedure as described above.

The antibody thus obtained may be modified in various ways, if the specific binding function of the protein or the like according to the present invention is preserved. Such modified antibodies are also included in the present invention. The modified antibodies include chimera and humanized antibodies.

The anti-SAKI antibody according to the present invention is used, for example, for detection of SAKI or the like (SAKI or the homologue) and isolation and purification of SAKI or the like. Use of a labelled antibody makes the detection above easier. Compounds used for antibody labelling include fluorescent dyes such as fluorescein, rhodamine, Texas Red, and Oregon Green; enzymes such as horseradish peroxidase, microperoxidase, alkaline phosphatase, and β-D-galactosidase; chemo- or bio-luminescent compounds such as luminol and acridine dyes; radioisotopes such as ³²P, ¹³¹I, and ¹²⁵I; biotin, and the like.

The anti-SAKI antibody according to the present invention can also be used as a drug to the diseases associated with SAKI or the like. For example, if there is an anti-SAKI antibody recognizing the site to be phosphorylated by M-period kinase and specifically binding to the site, it would be possible to form a constitutively phosphorylated SAKI state similar to amino acid substitution with aspartic acid (D) or glutamic acid (E) by modification of the site and thus to make the antibody show therapeutic action. Alternatively, if there is an anti-SAKI antibody specifically binding to dephosphorylated SAKI, it would be possible to inhibit nucleic acid-methylating action of the dephosphorylated SAKI by entrapping the activated SAKI and thus to make the antibody show therapeutic action. In particular, if there is an anti-SAKI antibody capable of binding to a methylation-related site, it would be effective in inhibiting methylation by dephosphorylated SAKI directly and thus show high therapeutic action. The term “disease,” as used in the present description, is the same as the term indicating abnormal state such as disease, sickness, or pathosis.

Another aspect of the present invention relates to an isolated SAKI-related nucleic acid. The aspect provides, for example, an isolated nucleic acid coding SAKI or the like (SAKI or the homologue), a nucleic acid that can be used as a probe for identifying the nucleic acid coding SAKI or the like and a nucleic acid that can be used as a primer, for example, for amplification or mutation of the nucleic acid coding SAKI or the like.

The term “nucleic acids” in the present description include DNAs (including cDNAs and genomic DNAs), RNAs (including mRNAs), DNA analogues, and RNA analogues. The nucleic acid according to the present invention is not limited, and may be single stranded or double stranded. It is preferably a double-stranded DNA. Codon degeneracy is also considered. Thus, a nucleic acid coding a protein may have any nucleotide sequence, if the desired expression product protein is obtained.

For example, the “nucleic acid coding SAKI (nucleotide sequence)”, as used in the present description, means a nucleic acid (nucleotide sequence) giving SAKI when expressed, and the nucleic acids include the nucleic acid having the nucleotide sequence corresponding to the amino acid sequence of the protein and also nucleic acids having an additional sequence not coding any amino acid added to the sequence (e.g., DNAs containing one or multiple introns).

In the present description, the term “isolated nucleic acid” specifically means a nucleic acid isolated from other nucleic acid copresent in the natural state, when the nucleic acid is a naturally present nucleic acid (e.g., human internal nucleic acid). However, the protein may contain some other nucleic acid components such as nucleic acid sequences neighboring in the natural state. For example, the “isolated nucleic acids” of a genomic DNA include, in its favorable state, substantially no other DNA components copresent in the natural state (including DNA sequences close in the natural state).

For example, in the case of a nucleic acid produced by genetic engineering technique such as cDNA molecule, the “isolated nucleic acid” is preferably a nucleic acid in the state substantially containing no cell components, culture solution and the like. Similarly in the case of a nucleic acid produced by chemical synthesis, the “isolated nucleic acid” is preferably a nucleic acid in the state containing substantially no precursors (raw materials) such as dDNTP, chemicals used in the synthesis process, or the like.

The “nucleic acid” simply described in the present description means a nucleic acid in the isolated state, unless specified otherwise.

The nucleic acid according to the present invention can be prepared into the isolated state, with reference to the sequence information disclosed in the present description or the attached sequence tables and by using a standard technique such as genetic engineering technique, molecular-biological technique or biochemical technique.

For example, the nucleic acid according to the present invention having the nucleotide sequence of SEQ ID No. 1 can be isolated by a hybridization method of using the nucleotide sequence or all or part of the complementary sequence as the probe. It can also be amplified and isolated by means of a nucleic acid amplification reaction (for example PCR) using a synthetic oligonucleotide primer designed to specifically hybridize with part of the nucleotide sequence. The oligonucleotide primer can be synthesized generally, easily for example by using a commercially available automatic DNA synthesizer.

In a favorable embodiment, the nucleic acid according to the present invention has a nucleotide sequence of SEQ ID No. 1 or 3. The nucleic acid having the nucleotide sequence of SEQ ID No. 1 is a full-length cDNA cloned as a sequence coding SAKI from a HeLa cell full-length cDNA library. On the other hand, the nucleic acid having the nucleotide sequence of SEQ ID No. 3 is a genomic DNA corresponding to the full-length cDNA (provided by Entrez Genome, NCBI, http://www.ncbi.nlm.nih.gov, Accession No. NC_{—}000005, Homo sapiens chromosome 5, complete sequence).

In another embodiment of the present invention, the invention provides DNA molecules having the nucleotide sequence of SEQ ID No. 1 but lacking the 5′-untranslated region or part thereof and also those lacking the 3′-untranslated region or one or more regions thereof (e.g., DNA only of coding regions (SEQ ID No. 4)). DNAs having an untranslated region different from the original untranslated region in the coding region are also included in the present invention, under the condition that it does not have any adverse effect on translation of the coding region.

In yet another embodiment, the present invention provides the nucleotide sequence coding the protein or the like according to the present invention or a nucleic acid that codes a protein having a function identical with that of the nucleotide sequence of SEQ ID No. 1, 3, or 4 but is different partially in nucleotide sequence (hereinafter, referred to also as “homologous nucleic acid”). Examples of the homologous nucleic acids include DNAs coding a protein that becomes a substrate for M-period kinase and has nucleic acid-methylating activity suppressed by phosphorylation, which has the nucleotide sequence coding the protein or the like according to the present invention or a nucleotide sequence containing substitution, deletion, insertion, addition, or inversion of one or more nucleotides, compared with the nucleotide sequence of SEQ ID No. 1, 3, or 4. The substitution or deletion may occur at multiple nucleotide sites. The “multiple nucleotide sites” means for example 2 to 40 bases, preferably 2 to 20 bases, and more preferably 2 to 10 bases, although it varies according to the position and the kind of the amino acid residues in the 3D structure of the nucleic acid-coding protein. Such a homologous nucleic acid is prepared, for example, by modifying the nucleotide sequence coding the protein or the like according to the present invention or the nucleic acid having a sequence of SEQ ID No. 1, 3, or 4 by genetic engineering so that there is substitution, deletion, insertion, addition, or inversion of bases at a specific site by using site-specific mutation method. Alternatively, the homologous nucleic acid can also be obtained by other methods such as UV irradiation.

Other examples of the homologous nucleic acids include nucleic acids containing a base difference caused by polymorphism such as SNP.

Another embodiment of the present invention relates to a nucleic acid having a nucleotide sequence complementary to the nucleotide sequence coding the protein or the like according to the present invention, or the nucleotide sequence of SEQ ID No. 1, 3, or 4. In yet another embodiment, the present invention provides a nucleic acid having the nucleotide sequence identical to the nucleic acid having a nucleotide sequence complementary to the nucleotide sequence coding the protein or the like according to the present invention or the nucleotide sequence of SEQ ID No. 1, 3, or 4 at a rate of at least 60%, preferably 70% or more, more preferably 80% or more, more preferably 90% or more, more preferably 95% or more, more preferably 99% or more, and most preferably 99.9% or more.

Yet another embodiment of the present invention relates to a nucleic acid hybridizing to the complementary chain of having the nucleotide sequence coding the protein or the like according to the present invention or the nucleotide sequence of SEQ ID No. 1, 3, or 4 under stringent condition. The “stringent condition” herein is a condition where a so-called specific hybrid is formed but non-specific hybrids are not formed. The stringent conditions are known by those who are skilled in the art, and such a condition can be determined, for example, according to Molecular Cloning (Third Edition, Cold Spring Harbor Laboratory Press, New York) or Current Protocols in Molecular Biology (edited by Frederick M. Ausubel et al., 1987). An example of the stringent condition is a condition of incubation at approximately 42° C. to approximately 50° C. by using a hybridization solution (50% formaldehyde, 10×SSC (0.15 M NaCl, 15 mM sodium citrate, pH 7.0), 5× Denhardt solution, 1% SDS, 10% dextran sulfuric acid, 10 μg/ml denatured salmon sperm DNA, 50 mM phosphate buffer (pH 7.5)) and subsequent cleaning by using 0.1×SSC and 0.1% SDS at approximately 65° C. to approximately 70° C. An example of still more favorable stringent condition is a condition of using a mixture of 50% formaldehyde, 5×SSC (0.15 M NaCl, 15 mM sodium citrate, pH 7.0), 1× Denhardt solution, 1% SDS, 10% dextran sulfuric acid, 10 μg/ml denatured salmon sperm DNA, and 50 mM phosphate buffer (pH 7.5)) as the hybridization solution.

In yet another embodiment of the present invention, it provides a nucleic acid (nucleic acid fragment) having part of the nucleic acid having a nucleotide sequence complementary to the nucleotide sequence coding the protein or the like according to the present invention or the nucleotide sequence of SEQ ID No. 1, 3, or 4. Such a nucleic acid fragment can be used, for example, for detection, identification, and/or amplification of the nucleic acid according to the present invention such as the nucleic acid having the nucleotide sequence of SEQ ID No. 1, 3, or 4. The nucleic acid fragment is designed, for example, to have at least a region hybridizing with a continuous nucleotide region in the nucleotide sequence of SEQ ID No. 1, 3, or 4 (e.g., of approximately 10 to approximately 100 nucleotide length, preferably approximately 20 to approximately 100 nucleotide length, and still more preferably approximately 30 to approximately 100 nucleotide length).

For use as a probe, the nucleic acid fragment may be labelled. For example, a fluorescent substance, an enzyme or a radio isotope can be used for labelling.

Yet another aspect of the present invention relates to a vector containing the nucleic acid according to the present invention (i.e., nucleic acid coding SAKI, part thereof, or the homologue thereof). In the present description, the term “vector” means a nucleus acid molecule that can transport an inserted nucleic acid, for example, into a target such as cell, and examples thereof include plasmid vectors, cosmid vectors, phage vectors, viral vectors (adeno viral vectors, adeno-associated viral vectors, retroviral vectors, herpes viral vectors, and the like).

A suitable vector is selected according to the application (cloning, protein expression) or to the kind of the host cell. Examples thereof include E. coli-hosted vectors (M13 phage or the mutant thereof, λ phage or the mutant thereof, pBR322 or the mutant thereof (e.g., pB325, pAT153, and pUC8), and others), yeast-hosted vectors (pYepSec1, pMFa, pYES2, etc.), insect cell-hosted vectors (pAc, pVL, etc.), and mammal cell-hosted vectors (pCDM8, pMT2PC, etc.).

The vector according to the present invention is preferably an expression vector. The “expression vector” is a vector capable of introducing its inserted nucleic acid into a desired cell (host cell) and leading to expression of the nucleic acid in the cell. An expression vector normally has a promotor sequence essential for expression of the inserted nucleic acid, an enhancer sequence for acceleration of expression, and others. An expression vector containing a selection marker may also be used. When such an expression vector is used, presence or absence of the introduced expression vector (and the degree) can be confirmed by using a selection marker.

Insertion of the nucleic acid according to the present invention into a vector, insertion of a selection marker gene (if needed), and insertion of a promotor (if needed) can be carried out, for example, by a standard recombinant DNA technique (well-known method of using restriction enzymes and DNA ligases seen in e.g., Molecular Cloning, Third Edition, 1.84, Cold Spring Harbor Laboratory Press, New York.).

Examples of the host cells include mammal cells such as of human, monkey, mouse, and rat (COS cell, CHO cell, etc.), microbial cells such as of E. coli, yeast cells, insect cells and others.

The other aspect of the present invention relates to a host cell containing the introduced nucleic acid according to the present invention (i.e., transformant). The transformant according to the present invention is preferably obtained by transfection or transformation by using the vector according to the present invention. The transfection or the like is carried out by calcium phosphate coprecipitation, electroporation (Potter, H. et al., Proc. Natl. Acad. Sci. U.S.A. 81, 7161-7165 (1984)), lipofection (Felgner, P. L et al., Proc. Natl. Acad. Sci. U.S.A. 84, 7413-7417 (1984)), microinjection (Graessmann, M. & Graessmann, A., Proc. Natl. Acad. Sci. U.S.A. 73, 366-370 (1976)) or the like.

The transformant according to the present invention can be used for production of SAKI or the like. Thus, another aspect of the present invention provides a method of producing SAKI or the like by using the transformant. The production method according to the present invention includes at least a step of culturing the transformant under a condition allowing production of SAKI or the like. Normally in addition to the step above, a step of recovering (isolating and purifying) the protein produced is also included. An example of the method of recovering the protein according to the present invention is a method of purifying the His-tag-SAKI produced by a known method in a suitable host cell such as E. coli by nickel column chromatography. Another method is a purification method by immunoprecipitation by using an antibody to SAKI or an antibody to the SAKI phosphorylated site, or the like. However, the invention is not limited thereto.

The transformant may be obtained not for production of SAKI or the like according to the present invention, but for example for the purpose of studying the behavior of the expressed SAKI or the like in a particular cell or the change in cellular state by expression of SAKI or the like in a particular cell (e.g., for therapeutic treatment). Alternatively, the transformant may be obtained for establishment of a transgenic animal (excluding human). Thus, the transformant according to the present invention may be used for establishment of a non-human transgenic animal. For example, it is possible to generate a transgenic animal by preparing, as a transformant, a fertile oocyte or an embryonic stem cell containing the nucleic acid coding SAKI or the like according to the present invention and proliferating the cell. In the present invention, such a non-transgenic animal is useful for studies on the functions of SAKI in vivo. The transgenic animal can be prepared, for example, by a microinjection method of injecting DNA directly into the anterior nucleus of fertilized egg, a method of using a retroviral vector, or a method of using ES cells. Hereinafter, the microinjection method will be described as an example of the method of producing a transgenic animal.

In the microinjection method, first, fertilized eggs are harvested from the fallopian tube of a female mouse after mating, and after culture of the eggs, a DNA construct (DNA coding the protein or the like according to the present invention) is injected into the anterior nuclei of the eggs. The DNA construct used preferably has a promotor sequence enabling efficient expression of the introduced gene. Examples of the promoters include chicken β-actin promotor, prion protein promotor, human AR promotor, neurofilament L-chain promoter, L7 promotor, cytomegalovirus promotor, and the like. The fertilized eggs after injection are transplanted in the fallopian tube of a pseudopregnant mouse, and the mouse is grown for a particular period after transplantation to give infant mice (FO). For confirmation of the adequate incorporation of the introduced gene into the chromosome of the infant mouse, DNA is extracted from the tail of the infant mouse and subjected for example to PCR method by using a promotor specifically binding to the introduced gene or to dot hybridization method by using a primer specifically binding to the introduced gene.

In the present description, the species of the “transgenic animal” is not particularly limited, but preferably a mammal animal, more preferably a rodent animal such as mouse or rat.

Yet another aspect of the present invention relates to application or use of SAKI or the relevant nucleic acid or the like. The aspect of the invention provides a method of determining the malignancy of cell (malignancy-determining method). In an embodiment of the malignancy-determining method according to the present invention, a step of determining the amount of SAKI (including phosphorylated SAKI) or the homologues (SAKI or the like) in the analyte cell isolated from the body is carried out. In yet another embodiment of the present invention, a step of determining the amount to the nucleic acid coding SAKI or the like in the analyte cell isolated from the body is carried out.

The “cellular malignancy” in the present description means the degree of cellular canceration. Thus, high-malignancy cells may be called cancer cells, while low-malignancy cells, normal cells. Alternatively, the “analyte cell” is a cell of which the malignancy is determined by the method according to the present invention. The analyte cell is isolated from the body. Accordingly, the present invention is applied to the analyte cell in the state immediately after isolation from the body. The phrase “isolated from the body” means a state where the analyte cells are completely separated from the components derived from the body tissue present in the analyte cells that is dissected for analysis. The analyte cells for use in the method according to the present invention are normally prepared in the state as they were in the body, i.e., in the state as bound to surrounding cells. The analyte cells may be used as they are separated (isolated) from the surrounding cells.

The analyte cells can be collected, for example, from a suspected cancer tissue. Specifically, part of a suspected cancer tissue may be collected by biopsy and the analyte cell-containing sample may be used in the method according to the present invention.

In the present description, the “cancer” is to be understood widely, and thus include carcinomata and sarcomata. Also in the present invention, the term “cancer” is the same as the term “tumor”.

On the other hand, “determination of the amount of SAKI or the like” is quantitative or qualitative determination of the absolute amount of the SAKI or the like present. The relative amount of SAKI or the like can be determined, for example, by reference to standard samples prepared according to the degree of canceration. The “determination of the amount of SAKI or the like” includes simple evaluation of presence or absence of SAKI or the like. Normally, presence or absence of SAKI or the like is studied first, and the amount thereof is determined then, if it is present. It is not always essential to determine the amount of SAKI or the like strictly quantitatively. For example, it is sufficient to determine the amount of SAKI or the like to a degree allowing determination of the malignancy of the analyte cell by comparison with the amounts of the SAKI standard used as the indicator of malignancy. The term “determination of the amount of the nucleic acid coding SAKI or the like” is to be understood similarly.

In the malignancy-determining method according to the present invention, the malignancy of the analyte cell is determined, based on the amount of SAKI or the like or the nucleic acid coding SAKI or the like, as determined in the step above. Specifically for example, a large detection amount may be determined as high malignancy of the analyte cell (specifically, for example as cancer cell). The detection amount obtained in the step above may be compared with the malignancy levels previously classified according to the detection amount. In this way, it is possible to rank the malignancy of analyte cell with standardized criteria.

The kind of the cancer to be analyzed by the malignancy-determining method according to the present invention is not particularly limited. Examples of the cancers to which the present invention is applicable include colon cancer, oral cancer, lung cancer, esophageal cancer, liver cancer, pancreas cancer, kidney cancer, bladder cancer, ureter cancer, prostate cancer, uterine cervix cancer, skin cancer, mammary gland cancer.

The method of “determining the amount of SAKI or the like” in the step above is not particularly limited, but preferably, an immunological staining method is used. The immunological staining method allows rapid and high-sensitivity determination of the amount of SAKI or the like. The operation is also simple and easy. Therefore, the load imposed on the patient during measurement of the amount of SAKI or the like is minimized.

An anti-SAKI antibody is used in the immunological staining method, and the amount of SAKI or the like is determined by using the binding efficiency of the antibody (binding amount) as an indicator. Specifically, the anti-SAKI antibody binding amount is determined, after a step of contacting an anti-SAKI antibody with an analyte cell. The amount of SAKI or the like determined in the analyte cell is calculated from the measurement results. Specifically, the method according to the present invention can be practiced according to the immunological staining method shown below.

Immunological staining of a body tissue is generally performed in the following steps (1) to (9). There are many literatures and books for reference on the immunological staining methods of body tissues (e.g., Sternberger L A, Hardy P H, Cuculis J J, et al.: The unlabelled antibody enzyme method of immunochemistry. Preparation and properties of soluble antigen-antibody complex (horseradish peroxidase anti horseradish peroxidase) and its use in identification of spirochates. J. Histochem. Cytochem 18: 315-333, 1970; Nakane P K and Kawaoi A: Peroxidase-labelled antibodies. A new method of conjugation. J. Histochem. Cytochem 22: 1084-1091, 1974. Guesdon J L, Ternynck T, Avrameas S: The use of avidin-biotin interaction in immunoenzymatic techniques. J. Histochem. Cytochem 27: 1131-1139, 1979; Hiroshi Tsutsumi Immunohistochemistry-biopsy diagnosis. Clinical Test 31 (Special Issue): 1330-1342, 1987; Keiichi Watanabe, Izuo Nakane Ed.: Enzyme Antibody Method, Gakusai Kikaku, 1992).

### (1) Fixation and Paraffin Embedding

A tissue surgically collected from the body is fixed, for example, with formalin, paraformaldehyde, or anhydrous ethyl alcohol. The tissue is then embedded in paraffin. Generally, the tissue is dehydrated with alcohol, treated with xylene, and then embedded in paraffin finally. The paraffin-embedded sample is sliced into a section having a desired thickness (e.g., 3 to 5 μm in thickness) and the section is spread on a slide glass. An alcohol-fixed sample, a dry enclosed sample, a frozen sample, or the like may be used instead of the paraffin-embedded sample.

### (2) Paraffin Removal

Generally, the sample is treated with xylene, alcohol, and purified water in that order.

### (3) Pretreatment (Antigen Activation)

The sample is, for example, heat-treated and/or pressurized as needed for antigen activation.

### (4) Removal of Endogenous Peroxidase

If a peroxidase is used as the labelling substance during staining, the sample is previously treated with hydrogen peroxide solution for removal of endogenous peroxidase activity.

### (5) Inhibition of Nonspecific Reactions

The section is treated with bovine serum albumin solution (e.g., 1% solution) for about several minutes to dozens of minutes for inhibition of nonspecific reactions. The step may be eliminated by carrying out the following primary antibody reaction by using a bovine serum albumin-containing antibody solution.

### (5) Primary Antibody Reaction

An antibody diluted to a suitable concentration is placed dropwise on the slice spread on the slide glass and left thereon for reaction for dozens of minutes to several hours. After reaction, the slice is washed with a suitable buffer solution such as phosphate buffer.

### (6) Addition of Labelling Reagent

The labelling substance frequently used is peroxidase. A drop of a peroxidase-bound secondary antibody is placed on the slice on slide glass and left thereon for reaction for dozens of minutes to several hours. After reaction, the slice is washed with a suitable buffer solution such as phosphate buffer.

### (7) Color Development Reaction

DAB (3,3′-diaminobenzidine) is dissolved in Tris buffer solution. Then, hydrogen peroxide solution is added. The section is immersed in the staining solution thus prepared for several minutes (e.g., 5 minutes) for color development. The section after color development is washed thoroughly with tap water for removal of DAB.

### (8) Nuclear Staining

Nuclear staining is performed in reaction with Mayer's hematoxylin solution for several seconds to dozens of seconds. The slice is washed with running water for color development (normally, for several minutes).

### (9) Dehydration, Penetration and Embedding

The slice is dehydrated with alcohol, penetrated with xylene, and finally, embedded in a synthetic resin, glycerol, a rubber sirup, or the like.

The kind or the origin of the anti-SAKI antibody used in the immunological staining method is not particularly limited, if it specifically binds to SAKI or the like. The anti-SAKI antibody may be a polyclonal antibody, an oligoclonal antibody (mixture of several to dozens of antibodies), or a monoclonal antibody. The IgG fraction in an antiserum obtained by animal immunization and also the antibodies purified by affinity chromatography by using an antigen can be used as the polyclonal or oligoclonal antibodies. The anti-SAKI antibody may be a fragment of an antibody such as Fab, Fab′, F (ab′)₂, scFv, or dsFv antibody.

Use of a labelled antibody as the anti-SAKI antibody permits direct determination of the amount of bound antibody, based on the amount of the label as indicator. Therefore, it is a method easier to practice. On the other hand, it also has problems that it demands an anti-SAKI antibody bound to a labelling substance and that the detection sensitivity is generally lower. It is thus preferable to use an indirect detection method, such as a method of using a secondary antibody bound to a labelling substance or a method of using a polymer of a secondary antibody and a labelling substance bound to each other. The secondary antibody is an antibody specifically binding to the anti-SAKI antibody, and, for example, when an anti-SAKI antibody is prepared as rabbit antibody, the anti-rabbit antibody is used. Labelled secondary antibodies for use to various antibodies such as of rabbit, goat, and mouse are commercially available (e.g., from Funakoshi Co., Ltd. and Cosmo Bio Co., Ltd.) and a labelled secondary antibody suitable for the anti-SAKI antibody used in the present invention can be used as it is properly selected.

On the other hand, when the analyte cellular malignancy is determined based on the amount of the nucleic acid coding SAKI or the like as indicator, a nucleic acid hybridizing with the nucleic acid coding SAKI or the like under stringent condition (i.e., nucleotide sequence coding SAKI or the like, nucleotide sequence of SEQ ID No. 1, 3, or 4, or nucleic acid having a part of a nucleotide sequence complementary thereto (see above) is used.

As the probe and the primer used in the malignancy-determining method according to the present invention, a suitable DNA or RNA fragment is used arbitrarily according to the detection method. The nucleotide length of the probe or the primer is not particularly limited if it is a length permitting the function thereof, but, considering selectivity, detection sensitivity and reproducibility, the primer nucleotide length is for example 10 bp or more, preferably 15 bp or more, specifically about 10 to 30 bp, and preferably about 15 to 25 bp. If it is a primer, it may contain some mismatching with the template sequence, if it can hybridize with and amplify the nucleid acid fragment to be amplified. The number of mismatching sites is for example 1 to several, preferably 1 to 5, and more preferably 1 to 3. If it is a probe, it may also contain some mismatching with the sequence to be detected in the range that does not affect detection.

The method of determining the amount of a specific nucleic acid is known in the art, and examples thereof include Southern hybridization method, Northern hybridization method, in-situ hybridization method, RT-PCT method and the like.

In yet another embodiment, the present invention provides a kit for carrying out the method. Use of the kit according to the present invention enables easier operation of the method in a shorter period of time. The kit according to the present invention contains various reagents suitable to the method applied. Specifically, in a method of detecting SAKI or the like (i.e., method of detecting the protein), a reagent specifically binding to SAKI or the like is used. Favorable examples of the reagent include anti-SAKI antibodies, but are not limited thereto. In a kit for detecting the binding amount of the anti-SAKI antibody directly, a labelled anti-SAKI antibody is used. On the other hand, in a kit by indirect detection method, an unlabelled anti-SAKI antibody is used. In such a case, a labelling substance-labelled secondary antibody (labelling secondary antibody) may be included in the kit. In the case of a kit containing a polymer of a secondary antibody and a labelling substance bound to each other for detection method, the polymer may be contained in the kit. The kit according to the present invention may contain one or more reagents and devices needed for antigen-antibody reaction and staining or immunostaining (e.g., formalin and paraffin for organ fixation and embedding, BSA for inhibition of nonspecific binding, color developing agent such as DAB, hematoxylin solution for nuclear staining, and others).

On the other hand, in a method of detecting the nucleic acid coding SAKI or the like (i.e., method of detecting the nucleic acid), nucleic acids (probe and/or primer) hybridizing with the nucleic acid under stringent condition is used. In such a case, the kit may contain one or more reagents and devices needed for hybridization (e.g., buffer solution, pH-adjusting reagent, and others) additionally.

Normally, the kit according to the present invention contains an attached instruction manual for use.

In yet another embodiment, the present invention provides a method of screening a compound effective to SAKI-related diseases. In the present description, the “SAKI-related diseases” are diseases caused by abnormal expression of SAKI (or its homologues), more specifically, diseases characterized by abnormality of expression amount of the proteins having the amino acid sequence of SEQ ID No. 2 (or their homologues) or abnormality of present amount of the nucleic acids having the nucleotide sequence of SEQ ID No. 1 or 3. The “abnormality” herein means a state of increase or decrease beyond a range of normal state. The “compound effective to SAKI-related diseases” is a compound effective in prevention or treatment of the SAKI-related diseases.

An embodiment of the screening method according to the present invention includes a step of examining whether the analyte compound can inhibit the binding between SAKI or the like and its phosphorylating enzyme M-period kinase (presence or absence of inhibition) and/or determining the degree of inhibition. If an analyte compound is found to inhibit the binding in the step, the compound is selected as an effective candidate drug. If particularly high inhibition is observed, the compound is considered to be a candidate drug of particular importance.

For example, the step is carried out in the following procedure. First, SAKI or the like and its phosphorylating enzyme M-period kinase are brought into contact with each other in the presence of the analyte compound (Step 1). Then, the amount of binding between SAKI or the like and the M-period kinase is compared with that when SAKI or the like and the M-period kinase are brought into contact in the absence of the analyte compound (Step 2). If the latter binding amount is larger, the analyte compound is considered to be inhibitory to binding between SAKI or the like and the M-period kinase.

In yet another embodiment, the screening method according to the present invention includes a step of examining whether the analyte compound inhibits suppression of the nucleic acid-methylating activity of SAKI or the like and the nucleic acid-methylating activity of phosphorylated SAKI (presence or absence of inhibition) and/or of determining the degree of inhibition. For example, the step may be carried out in the following procedure. First, phosphorylated SAKI and nucleic acid substrate are brought into contact with each other in the presence of an analyte compound and a methyl group donor (Step 21). Then, the degree of methylation on the nucleic acid substrate is compared with that when the phosphorylated SAKI and the nucleic acid substrate are brought into contact similarly to above in the absence of the analyte compound (Step 22). If the latter degree of methylation is larger, the analyte compound is considered to effective in inhibiting the nucleic acid-methylating activity of SAKI or the like.

The Step 1 is carried out, for example in the following procedure. First, phosphorylated SAKI is generated (phosphorylation reaction: Step 1-1). Then, an analyte compound, a methyl group donor, the phosphorylated SAKI, and a nucleic acid substrate are mixed and allowed to react with each other (methylation reaction: Step 1-2). In the embodiment, phosphorylation of SAKI and methylation reaction by phosphorylated SAKI are carried out separately in mutually independent steps. The methylation reaction is carried out, for example under the following condition.

Temperature: approximately 37° C., pH: approximately 7.8, and reaction period: approximately 30 minutes.

Alternatively, the Step 21 may be carried out in the following procedure. Specifically, an analyte compound, a methyl group donor, a SAKI, kinase, and a nucleic acid substrate are mixed and allowed to react with each other (phosphorylation and methylation reactions). In the present embodiment, generation of phosphorylation of SAKI and methylation reaction of phosphorylated SAKI proceed in a single step. Thus, the operation is simplified and the reaction period is shortened. In addition, the reaction is closer to the environment in the body, and thus, the detection result reflects the phenomena of the analyte compound in the body more favorably, thus allowing selection of more effective compounds.

In the method above, a M-period kinase is used favorably as the kinase for phosphorylation of SAKI. The methyl group donor favorably used is methionine or a methionine derivative, but is not limited thereto. The methyl group donor may be labelled (e.g., with radioactive label).

For easy and highly-sensitive detection of the methylating activity, a reagent containing many cytosines is used favorably as the nucleic acid substrate. Specifically, Poly-dI:dC may be used favorably, for example. Such nucleic acid reagents are commercially available (e.g., Roche), and are easily purchasable.

Organic compounds with various molecular sizes (nucleic acids, peptides, proteins, lipids (simple lipids, complex lipids (phosphoglycerides, sphingolipids, glycosylglycerides, cerebrosides, etc.), prostaglandins, isoprenoids, terpenes, steroids, etc.) or inorganic compounds can be used as the analyte compounds in the screening method according to the present invention. The analyte compound may be a natural product-derived compound or a synthetic compound. In the case of the latter compound, an efficient screening system can be established, for example, by using a combinatorial synthetic technique. Cell extracts, culture supernatants, or the like may be used as the analyte compounds.

The present invention will be described in more detail based on examples given below, but the invention is not meant to be limited by these.

### EXAMPLES

### Example 1

### 1. Identification of SAKI

### 1-1. Preparation of an Antibody Recognizing Serine Site Phosphorylated by AIM-1 and Discovery of Protein Having a Molecular Weight of Approximately 100 kDa

C. D. Allis and the research group reported in 2000 that yeast and nematode Ipl1's are M-period phosphorylating enzymes of H3 histone (Hsu, J. Y. et al.: Mitotic phosphorylation of histone H3 is governed by Ipl1/aurora kinase and Glc7/PPI phosphatase in budding yeast and nematodes. Cell 102: 279-291, 2000.). The inventors have considered that AIM-1 was the kinase in animal cells analogous in function to Ipl1, which is a single enzyme only present in yeast, soon after the report by C. D. Allis et al., and demonstrated that the kinase for the M-period H3 histone in animal cells was AIM-1, based on structural comparison with an analogous kinase Aik (currently, called Aurora A) discovered by Okano et al. in Gifu University in 1996 when the AIM-1 was discovered, and also on the results obtained in incorporation test into yeasts and comparison of localization of proteins in cell by using antibodies. The presence of AIM-1 was considered to be similar in situation in that there were multiple Cdc2 enzymes in animal cells although only a single enzyme is present in yeast and, among the enzymes, G2-period HuCdc2 was recognized as the Cdk1. However, there is described a completely different view in E. A. Nigg's review (Nigg, E. A.: Mitotic kinases as regulators of cell division and its checkpoints. Nat. Rev. Mol. Cell. Biol. 2: 21-32, 2001.).

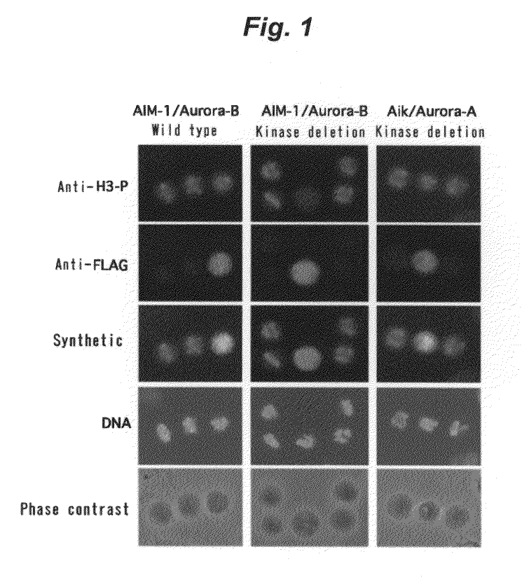

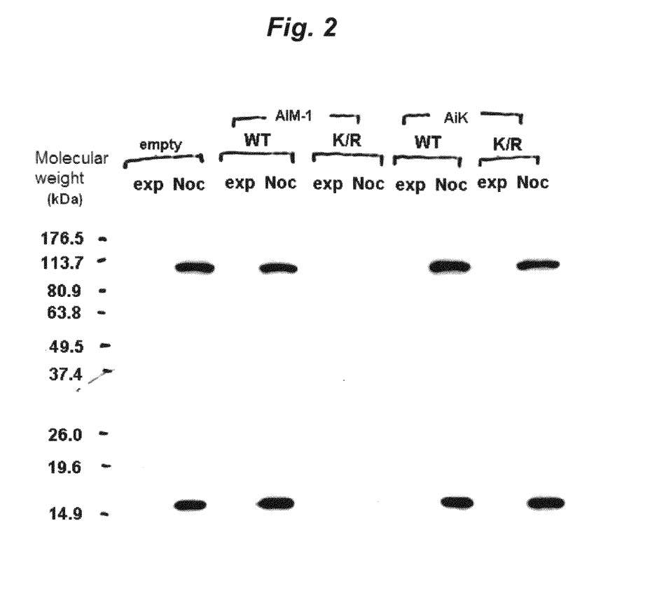

In the inventors' preliminary studies (in-vitro phosphorylation experiments), both AIM-1 and Aik phosphorylated H3 histone. Based on these results, the inventors tried to identify an intracellular responsible enzyme by the following method. First, a polyclonal antibody specifically recognizing a phosphorylation site (Ser10) of H3 histone was prepared. Specifically, a rabbit was immunized by using a bovine albumin-bound complex of 7 to 20th synthetic peptide of H3 histone having the 10th serine phosphorylated (ARKS*TGGKAPRKQL (S*=Phosphorylated serine): SEQ ID No. 5) as antigen to obtain a specific antibody of M-period phosphorylation site of H3 histone. For evaluation of the phosphorylation state in cell, a FLAG-tagged wild AIM-1 and an Aik-expressing vector, and a kinase-deficient mutant protein-expressing vector having the ATP-binding site substituted from lysine to alanine were introduced into HeLa cell, and immunostaining was performed by using an anti-FLAG antibody, DNA staining (chromosomally condensed M-period cells identified with DAPI), and an anti-H3 histone Ser 10 phosphorylation site antibody. As a result, AIM-1 was found to be a M-period phosphorylating enzyme of H3 histone (FIG. 1). It was also observed by immunofluorescent antibody method and also analyzed by immunoblotting by using the anti-H3 histone Ser-10 phosphorylation site antibody. As a result, it was confirmed that phosphorylation of H3 histone in the region of approximately 16 kDa was suppressed by expression of the AIM-1 kinase-deficient mutant protein, and that there was a protein that is seemingly phosphorylated by AIM-1 in the region close to 100 kDa (FIG. 2).

### 1-2. Identification of a 100 kDa Protein by Proteomics by Using Antibody Recognizing Phosphorylated Serine Site by AIM-1

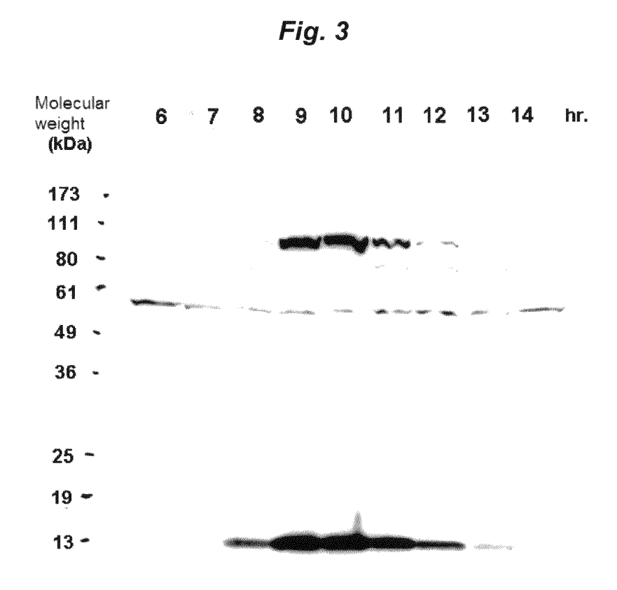

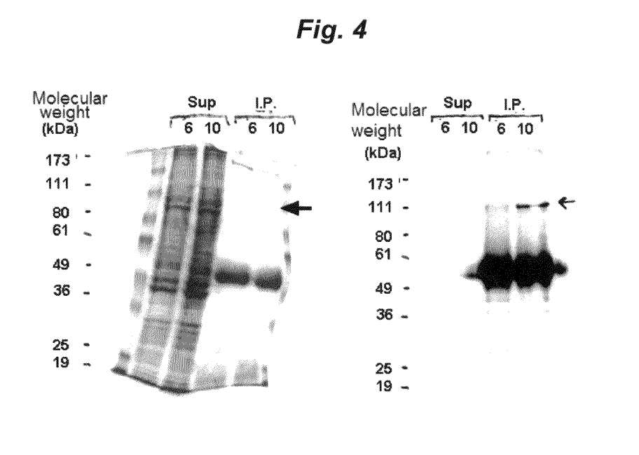

For identification of the protein having a molecular weight of approximately 100 kDa that is detected by the antibody recognizing the serine site phosphorylated by AIM-1, immunoprecipitation by using an antibody recognizing the phosphorylated serine site was performed. First, HeLa cell was synchronized by double-thymidine treatment method, and phosphorylation of the desired protein of approximately 100 kDa and cell cycle-dependent kinetics of phosphorylation of H3 histone were studied (FIG. 3). After initiation of culture of the synchronized cell, the cells after 10 hours were collected for recovery of the phosphorylation protein, while the cells after 6 hours were regarded not to contain the phosphorylated protein. Proteins collected from the cells at each collection time were subjected to immunoprecipitation experiment by using an antibody recognizing the serine site phosphorylated by AIM-1. As a result, it was found that the samples after synchronized culture for 10 hours contained an approximately 100 kDa protein that could be immunoprecipitated (FIG. 4, left column). The protein was visually recognizable after BPB staining (FIG. 4, right column). Then, HeLa cells (on 100 10-cm dishes) used as starting material 10 hours after synchronized release were subjected to immunoprecipitation by using an antibody recognizing the phosphorylated serine site, and the immunoprecipitated protein of approximately 100 kDa was separated from the gel, and the partial amino acid sequence was analyzed by Edman degradation method.

### 1-3. Cloning of Gene of 100 kDa Protein (SAKI)

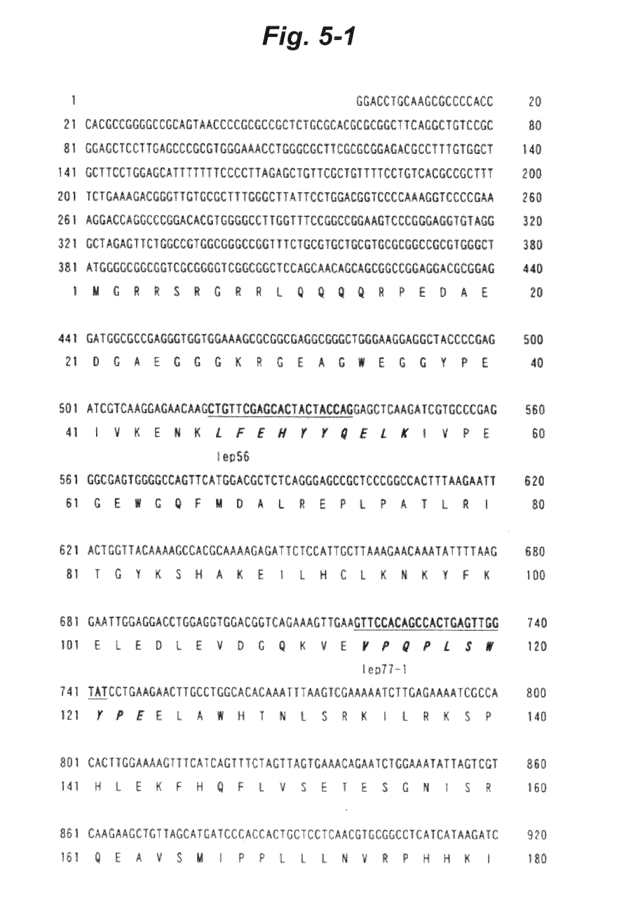

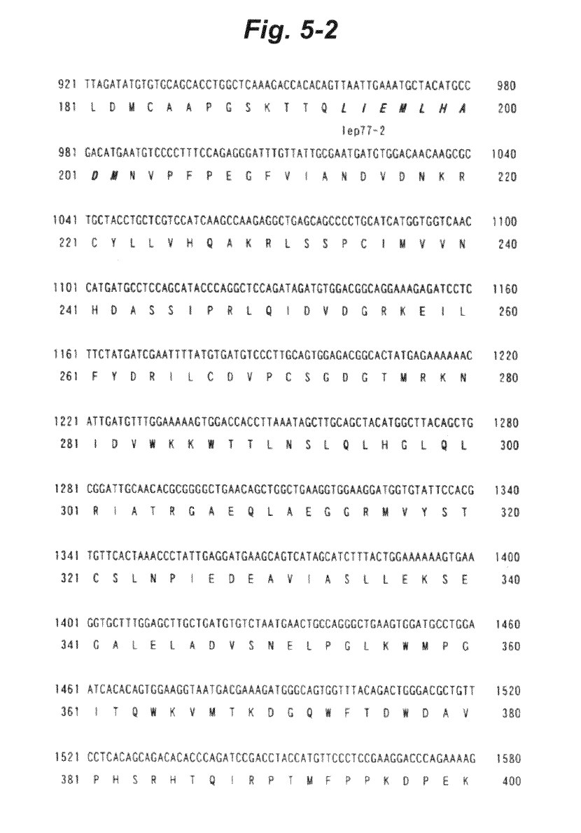

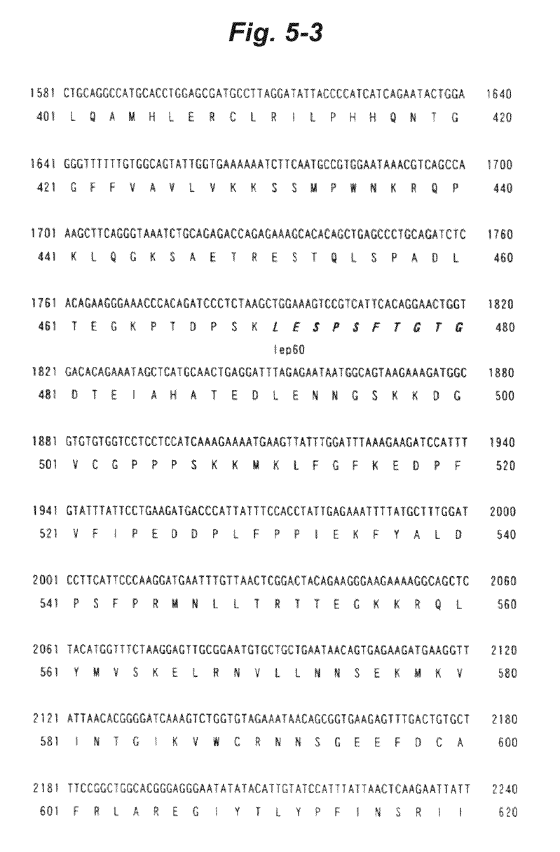

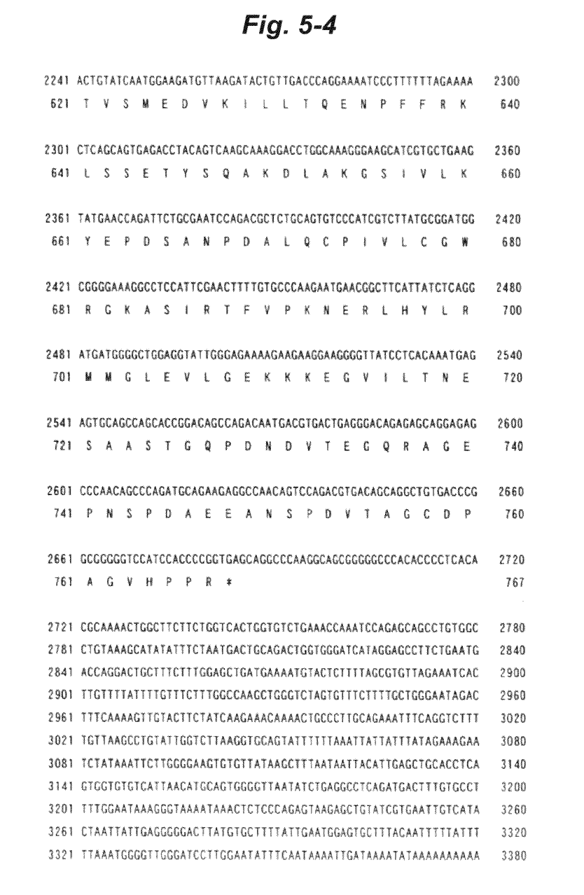

The partial amino acid sequences of four peptide fragments in the immunoprecipitated approximately 100 kDa protein were determined (lep56: LFEHYYQELK (SEQ ID No. 6), lep77-1: VPQPLSWYPE (SEQ ID No. 7), lep77-2: LIEMLHADM (SEQ ID No. 8), and lep60: LESPSFTGTG (SEQ ID No. 9)). After homology search by BLAST, only the lep60 region was completely identical with the amino acids coded by BC001041 cDNA sequence, but other three fragments did not have matched coding sequences. Thus, mRNA sequences corresponding to lep56 and lep77-1 were obtained from dbEST, and 5′-RACE primers having a region corresponding to the amino acid sequence (antisense sequence 5 corresponding to lep56, -CTGGTAGTAGTGCTCGAACAG-3′ (SEQ ID No. 10) or to an antisense sequence 5 corresponding to lep77-1, -ATACCAACTCAGTGGCTGTGGAAC-3′ (SEQ ID No. 11)) were prepared, and 5′-RACE was performed by using mRNA of HeLa cell. Then, the sequence of the 5′-RACE product cDNA fragments obtained were determined and the full-length cDNAs were cloned by colony-hybridization method from HeLa full-length cDNA library, by using the fragments. The open leading frame (SEQ ID No. 4)) in the approximately 3380-bp cDNA obtained (SEQ ID No. 11) coded an amino acid sequence (SEQ ID No. 2) of estimated 767 residues (FIGS. 5-1 to 5-4). The protein estimated from the coding sequence was designated as SAKI.

### 1-4. Identification of Phosphorylation Site of SAKI Protein

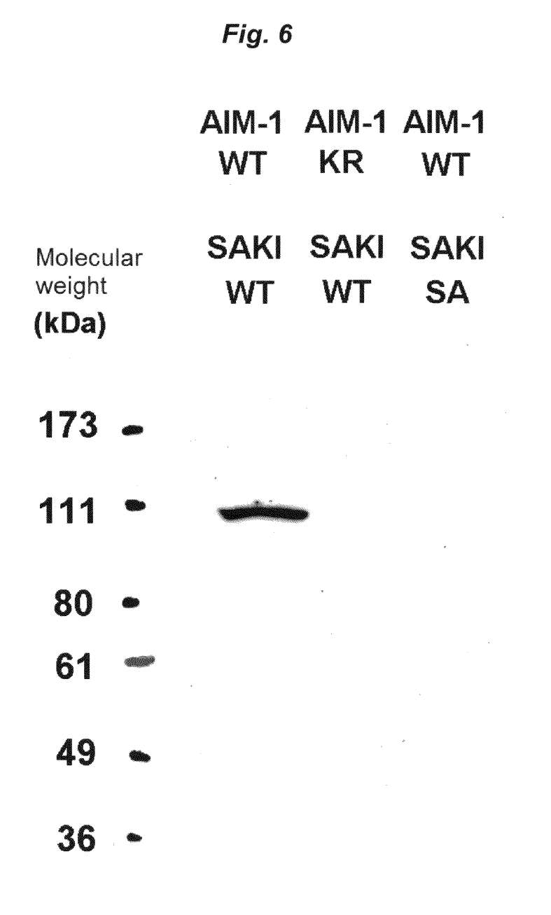

The amino acid sequence (RKS) of the epitope region (around 10th serine) of the anti-H3 histone Ser-10 phosphorylation site antibody (antibody recognizing the serine site phosphorylated by AIM-1) was present in the estimated SAKI amino acid sequence (137-139 amino acid residue region in SAKI). In preliminary experiments, a SAKI gene product lacking N-terminal 1-157 amino acid residues (Δ1-157-SAKI) and a SAKI gene product lacking C-terminal 541-767 amino acid residues (Δ541-767-SAKI) were expressed in HeLa cell forcedly, and subsequent immunoblotting experiment of the M period-harvested cells after Nocodazole treatment showed that the phosphorylation site is present in the N-terminal 1-157 amino acid residues. Therefore, a mutant gene (SAKI-SA) of SAKI with its 139th serine residue substituted with alanine was prepared and expressed in HeLa cell, and the cells in the M-period phosphorylation state after Nocodazole treatment were analyzed by immunoblotting by using an antibody recognizing the serine site phosphorylated by AIM-1, to show that SAKI phosphorylation in the SAKI-SA-expressing cell was suppressed (FIG. 6). The results indicated that the SAKI's phosphorylation site by AIM-1 was the 139th serine residue.

### 1-5. Homology Search of SAKI Protein

The full length of the estimated amino acid sequence showed that SAKI was structurally similar to a nucleolus protein belonging to NOL1/NOP2/sun family. However, it was a novel protein different from the human protein, Human NOL1 120-kDa protein already known. Updated database search showed that it was registered on Dec. 23, 2003 as a theoretical coding protein estimated from its genome (hypothetical protein FLJ20303, Accession: NP_{—}060225) in a database (Entrez Protein, NCBI, http://www.ncbi.nlm.nih.gov).

### Example 2

### 2. Distribution of SAKI Expression

### 2-1. Preparation of Anti-SAKI Antibody

(1) Preparation of an Antibody with C-Terminal Synthetic Peptide (Anti-C-Terminal SAKI antibody)

A synthetic peptide (GCDPAGVHPPR: SEQ ID No. 12) identical with the amino acid sequence of C-terminal region of SAKI was prepared, a rabbit was immunized with the peptide as antigen, and the antiserum obtained was purified by affinity chromatography, to give a polyclonal antibody.

### (2) Preparation of Antibody to Full-Length SAKI as Antigen

Separately from the C-terminal-recognizing antibody, a His-tag N-terminal-labelled SAKI full-length protein in E. coli was His-purified by pRSET, and a rabbit was immunized with the purified protein, to give an antibody to the full-length SAKI (anti-full-length SAKI antibody).

### 2-2. SAKI Expression Profile (Dependency on Cell Cycle and Organ-Specific Distribution)

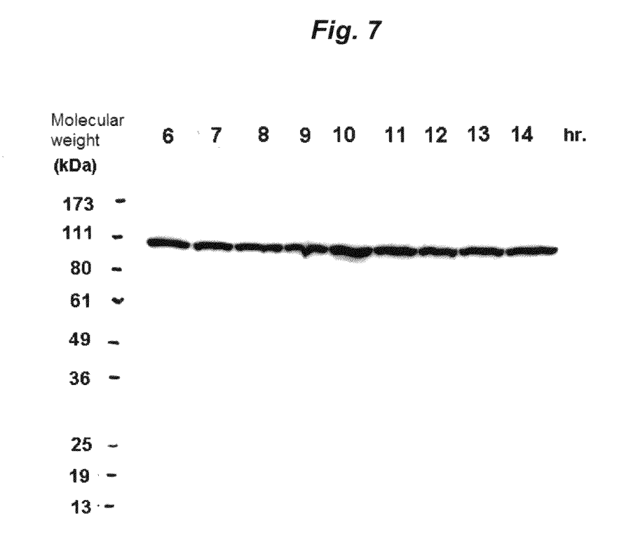

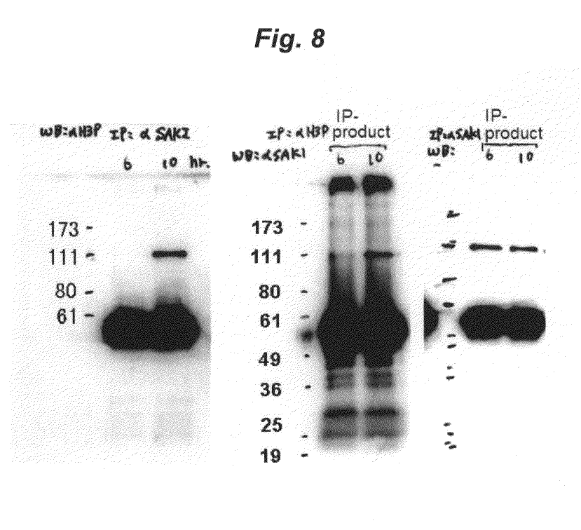

The following finding on the cell-cycle dependency of SAKI expression was obtained. Specifically, the SAKI expression pattern of a synchronized HeLa cell (see FIG. 3) was examined by immunoblotting with a C-terminal SAKI antibody, to show that the expression level of SAKI did not fluctuate throughout the cell cycle (FIG. 7). Immunoprecipitation experiment of the synchronized HeLa cells 6 hours after release (interphase) and 10 hours after release (M period) showed that the SAKI antibody immunoprecipitate 100-kDa protein was phosphorylated specifically in M period (FIG. 8).

### 2-3. Intracellular Localization by Immunostaining

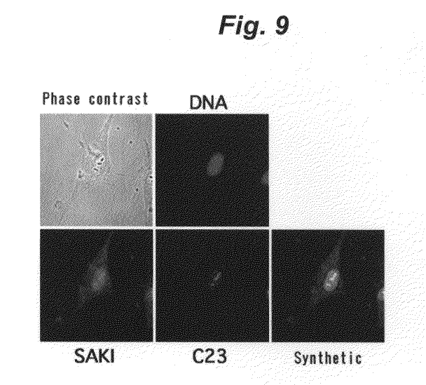

Immunostaining was carried out for intracellular localization of SAKI. As a result, dyeing affinity of SAKI in interphase cell was identical with the dyeing affinity of the nucleolus region and the nucleolus protein C23 in the phase-contrast cell image, indicating that SAKI was localized in nucleolus (FIG. 9). SAKI was observed extrachromosomally in M-period cell (data not shown). The nucleolus structure disappears in M period, but staining test results showed that the SAKI protein seemingly moved out of the nucleus (normal human fibroblast (hereinafter, NHDF) in FIG. 9), and staining of SAKI was observed also in the nucleolus region of HeLa cell. However, there was no distinct localization, such as that of NHDF cell, observed in HeLa cell, probably because of high expression of SAKI in HeLa cell.

### Example 3

### 3. Estimated Function of SAKI in Cell

3-1. rRNA Maturation-Activating Activity of SAKI Protein (SAKI as a Rate-Determining Enzyme of RNA Metabolism)

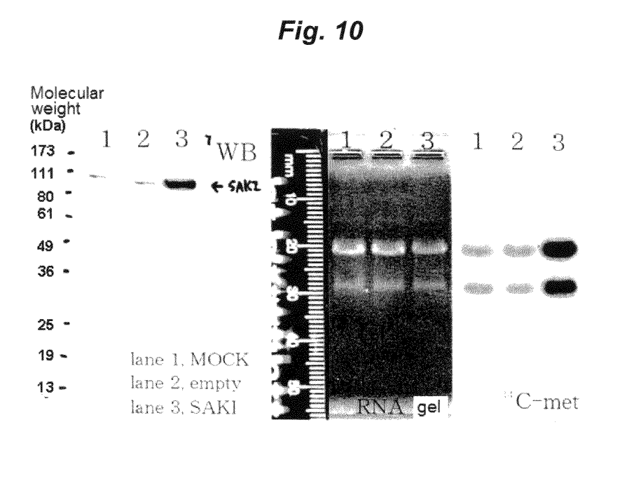

Structural similarity and localization in nucleolus in cell suggested that SAKI was a methylating enzyme involved in the process of rRNA maturation. For that reason, a SAKI gene-expressing vector was transfected into HeLa cell, the cell was cultured in a medium containing L-[methyl-¹⁴C]methionine as methyl group donor for 18 hours, the total RNA was recovered and subjected to electrophoresis in RNA gel and then to RNA blotting, and incorporation of ¹⁴C-labelled methyl group was studied by autoradiography. The results showed that overexpression of SAKI lead to increase in the amount of ¹⁴C-labelled methyl group incorporated into rRNA (FIG. 10). Thus, SAKI was considered to have an activity to methylate rRNA in cell.

### 3-2. Physiological Significance of M-Period-Phosphorylated SAKI Protein

The facts that SAKI is localized in interphase nucleolus with nucleolus proteins B23 and C23 (FIG. 9) and that immunoprecipitation of these proteins resulted in coprecipitation suggested formation of a complex. On the other hand, SAKI is phosphorylated before the M period. B23 and C23 are known to be phosphorylated in the same period. It is also known that nucleolus structure disappears simultaneously with disappearance of the nuclear membrane during transition from G2 to M period, but the behaviors of phosphorylated C23 and phosphorylated B23 are different in the M period when the nucleolus structure is lost. The amounts of the proteins SAKI, C23, and B23 expressed in the cell after Nocodazole treatment are not different from those in the interphase period. However, differently from the interphase, B23 and C23 are both more resistant to immunoprecipitation with the SAKI protein, and observation of these proteins in cell in M period showed different localization. Localization of the SAKI protein overlapped partially that of C23, and, similarly in immunoprecipitation experiment, SAKI seemed to have partial interaction with C23 in the M period. SAKI is located extrachromosomally similarly to C23 in the M period.

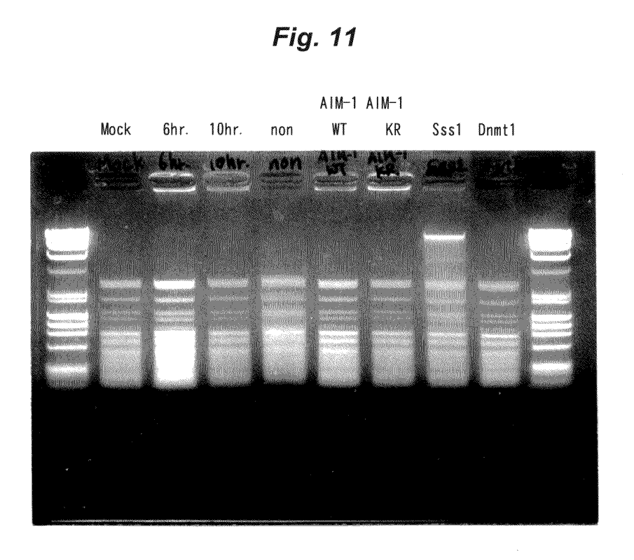

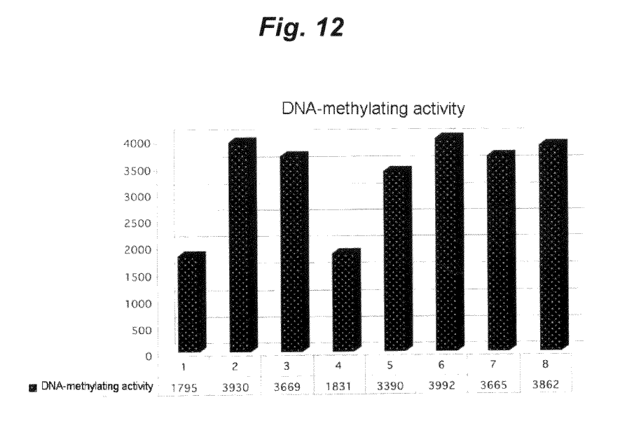

Separately, the DNA-methylating activity in vitro was determined by using an active-form AIM-1 produced in E. coli as the enzyme. As a result, there was no homo-methylating activity, as determined by using λDNA, observed in SAKI (normally, eucaryotes have no such activity) (FIG. 11). In addition, analysis of hemi-methylating activity, as determined by using polydI:dC as a substrate, showed a hemi-methylating activity in an E. coli-produced AIM-1-WT protein similar to the degree of human Dnmt1, but no increase in the hemi-methylating activity was observed by addition of the SAKI protein (FIG. 12), and there was no hemi-methylating activity by the SAKI protein alone (data not shown). Namely, SAKI had no such a DNA-methylating enzyme activity.

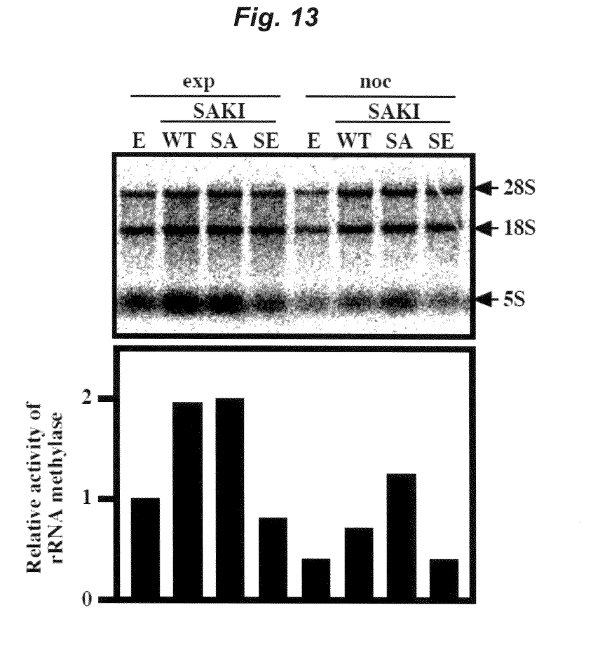

On the other hand, for analysis of the significance of SAKI phosphorylation, vectors expressing SAKI-WT, SAKI-SA (the 139th serine substituted with alanine), and SAKI-SE (the 139th serine substituted with glutamic acid) were incorporated into a HeLa cell, and the cell was labelled with 1 μCi/ml of L-[methy1-¹⁴C]methionine for 18 hours. A group (exp) in the logarithmic growth period during labelling and a group (noc) treated with 200 ng/ml of Nocodazole were processed for isolation of rRNA, subjected to RNA-gel electrophoresis and then, RI detection with BAS2000. The results are summarized in FIG. 13. The top chart in the figure shows the results of BAS2000, while the bottom chart shows quantitative amount of the methylated product. Lane E represents an empty vector (only vector), lane WT represents SAKI-WT, lane SA represents SAKI-SA, and lane SE represents SAKI-SE respectively.

As a result, similarly to the results shown in FIG. 10, there was increase in rRNA-methylating enzyme activity observed in wild-type (WT) and nonphosphorylated (SAKI-SA) cells (exp) in the logarithmic growth period. However, with constitutive phosphorylated SAKI (SAKI-SE), there was observed inhibition of the rRNA-methylating enzyme activity.

On the other hand, in the M-period (noc) cells, there was almost no difference in the rRNA-methylating enzyme activity between the empty vector cells (E) and the constitutive phosphorylated cells (SAKI-SE). The wild-type (WT) cells showed some increase in rRNA-methylating enzyme activity. In contrast, in SAKI-SA cells, similarly to the level in the logarithm growth period, there was observed increase in rRNA-methylating enzyme activity.

The results above indicate that SAKI does not have DNA-methylating enzyme activity but has rRNA-methylating activity, and the rRNA-methylating enzyme activity is suppressed by phosphorylation control in the M period.

### Example 4

### 4. SAKI-Phosphorylating Enzyme

SAKI is a protein identified with a polyclonal antibody recognizing the sequence of H3 histone phosphorylated at the 10th serine, and SAKI is also phosphorylated in the M period in cell cycle similarly to H3 histone. The enzyme phosphorylating the 10th serine of H3 histone in M period is the AIM-1/Aurora-B isolated and identified from an animal cell by the inventors. For that reason, SAKI was considered to be phosphorylated by AIM-1.

The experiment data shown in FIGS. 2 and 6 indicate that, “in AIM-1/Aurora-B cells, expression of the kinase-deficient K/R gene leads to suppression of phosphorylation H3 histone and also of SAKI”, and the results also were considered to suggest that “SAKI is a substrate phosphorylated by AIM-1/Aurora-B in the M period, similarly to H3 histone.”

Further for confirmation, it was examined whether SAKI phosphorylation in HeLa cell synchronized by double-thymidine treatment was influenced by Aurora kinase inhibitor treatment, by immunoblotting by using an antibody recognizing the serine site phosphorylated by AIM-1.

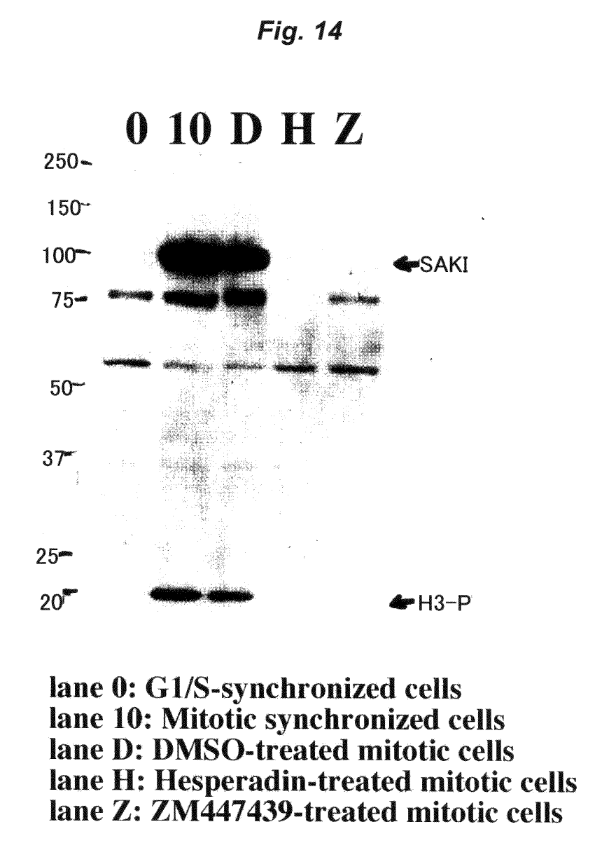

The HeLa cell synchronized to G1/S period (hour: 0) by double-thymidine treatment advanced in its cell cycle synchronously after release and enters into the M period after 10 hours. Both H3 histone and SAKI were not phosphorylated at hour 0 (FIG. 14, lane 0). On the other hand, H3 histone and SAKI were both phosphorylated in cells synchronized in M period after 10 hours (FIG. 14, lane 10).

The culture medium for the synchronized cell was exchanged after 8 hours, and AIM-1 was treated with a drug-dissolving solvent DMSO, an Aurora-inhibiting low-molecular-weight compound Hesperadin, or ZM447439. Both phosphorylation of H3 histone and phosphorylation of SAKI were suppressed in Hesperadin- or ZM447439-treated group (FIG. 14, lanes H and Z), compared to that in the drug-untreated control, DMSO-treated group (FIG. 14, lane D).

The results shown in FIGS. 2 and 6 as well as in FIG. 14 demonstrated that SAKI was a substrate for AIM-1.

### Example 5

### 5. Expression of SAKI in Various Human Cancers and its Application

### 5-1. Comparison of Expression Between in Cancer Cell and Normal Cell by Immunoblotting by Using Anti-SAKI Antibody

### (1) Expression in Normal Human Organ Tissues

A protein extraction sample of each organ was prepared, and expression of SAKI in each human internal organ was determined by immunoblotting method. As a result, the expression thereof was observed in testis, thyroid gland, salivary gland, tracheae, lung, duodenum, gingival epithelia, and lingual epithelia; expression at a trace level was observed in kidney, small intestine, spleen, bladder, adrenal gland, and tongue muscle; but no detectable expression was observed in artery, liver, colon, stomach, esophagus, or heart (FIG. 15).

### (2) Expression Pattern in Cell Lines

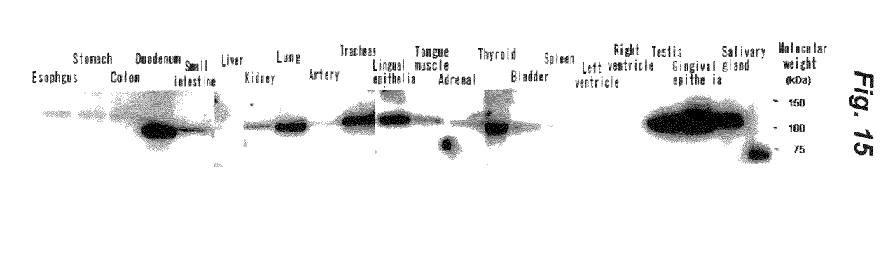

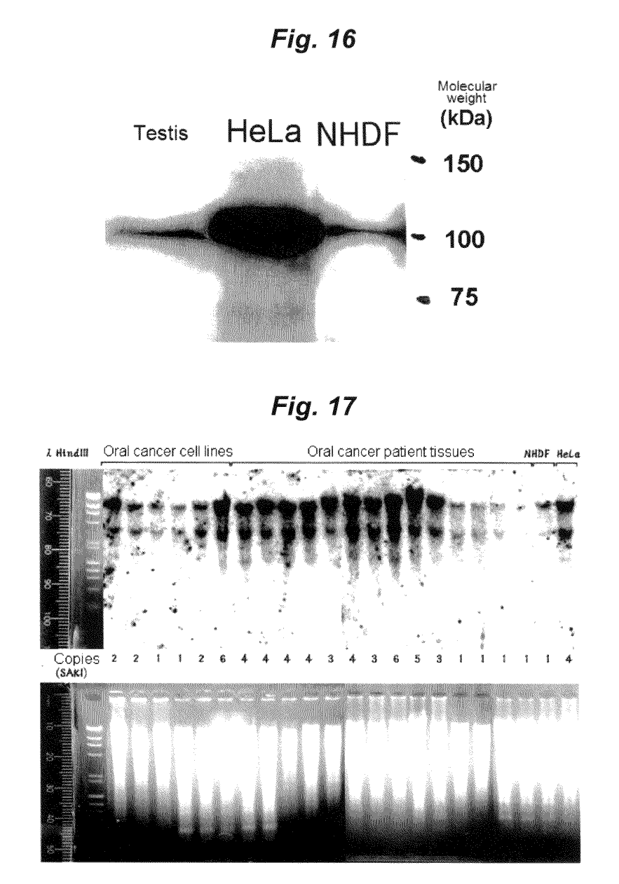

Higher expression of SAKI was observed in HeLa cells, compared to that in NHDF cells (FIG. 16). High expression of SAKI was observed in various colon cancer cell lines and all oral-cavity squamous cell carcinoma cell lines (data not shown).

### 5-2. Analysis of Clinical Cases by Genome Southern Blotting (Amplification of 5p15.32 SAKI Gene is Observed in Some Cancer Cases)

The SAKI gene was found to be mapped in human chromosome 5p15.32 by search of human-genome information. The region close to 5p15.3 is a region wherein the gene is amplified frequently in human cancers. Because of high expression of SAKI protein observed in cancer cell lines, the number of the SAKI gene copied was determined by genome Southern blotting. As a result, gene amplification was observed in HeLa cells wherein SAKI was highly expressed (FIG. 17). Clinical autopsy tissues of human oral cancer were studied and there were observed some tissues with amplified gene expression (FIG. 17). It was found that cancer-related high expression of SAKI was occasionally accompanied by gene amplification.

### 5-3. Comparison of Expression Between in Human Cancer Tissues and Normal Tissue by Immunostaining by Using Anti-SAKI Antibody

Pathologic samples of cancer tissue regions of human cancer patients were observed by immunohistologic staining by using an anti-C-terminal SAKI antibody. Immunohistologic staining (by high-sensitivity method (CSA method)) was performed in the following procedure.

### (1) Fixation, Paraffin Embedding, and Slicing

A surgically removed tissue is fixed in 10% neutrally buffered formalin solution for one to several days, dehydrated and then embedded in paraffin. It is then sliced into a section of approximately 5 μm in thickness, and the section is spread on a silane-coated slide glass.

### (2) Paraffin Removal

The paraffin is removed with xylene and a descending series of ethanol.

### (3) Blocking of Endogenous Peroxidase

The section is immersed in 0.3% H₂O₂/100% methyl alcohol for 30 minutes for inhibition of endogenous peroxidase activity.

### (4) Antigen Activation

It is then immersed in citrate buffer solution at pH 6.0, and ultrasonicated (5 minutes×3 times) for activation of the antigens in the section.

### (5) Blocking of Nonspecific Reactions

The section is washed with water and immersed in PBS, and a drop of a casein-containing nonspecific reaction-blocking reagent was placed on the section (room temperature, 10 minute).

### (6) Primary Antibody Reaction

A drop of a solution of an anti-SAKI C-terminal polyclonal antibody diluted 400 to 1,600 times with PBS is put on the section dropwise for reaction (4° C., overnight). The section is then washed with Tween 20-containing Tris/hydrochloric acid buffer solution (TBST).

(7) Reaction with Biotin-Labelled Secondary Antibody

A drop of biotin-labelled anti-rabbit immunoglobulin is placed on the section for reaction (room temperature, 15 minute). The section is then washed with TBST.

### (8) Reaction of Streptavidin-Biotin Complex

A drop of a streptavidin-biotin complex is placed on the section for reaction (room temperature, 15 minute), and the section is washed with TBST.

### (9) Reaction of Amplification Reagent

A drop of an amplification reagent (biotin-labelled tyramide) is placed on the section for reaction (room temperature, 15 minute). Then, the section is washed with TBST.

### (10) Reaction of Peroxidase-Labelled Streptavidin

A drop of a peroxidase-labelled streptavidin was placed on the section for reaction (room temperature, 15 minutes). The section is then washed with TBST.

### (11) Color Development

The section was dyed with DAB (5 to 20 minutes) and then, washed with water.

### (12) Contrast Staining

The nucleus is stained with hematoxylin (1 minute) and then the section is washed with water.

### (13) Encapsulation

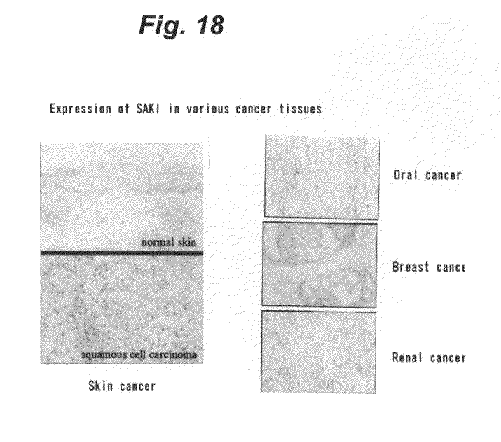

The section is dehydrated with an ascending series of ethanol, penetrated with xylene, and encapsulated. In this way, clear and distinct SAKI staining of cancer tissues were possible (FIG. 18), indicating that the method is applicable to diagnosis.

### 5-4. Evaluation of Usefulness and Significance of the Anti-SAKI Antibody in Human Cancer Diagnosis

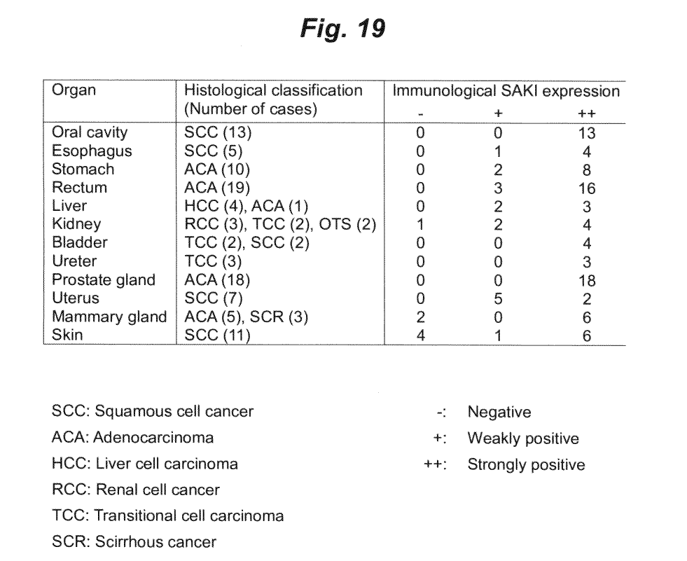

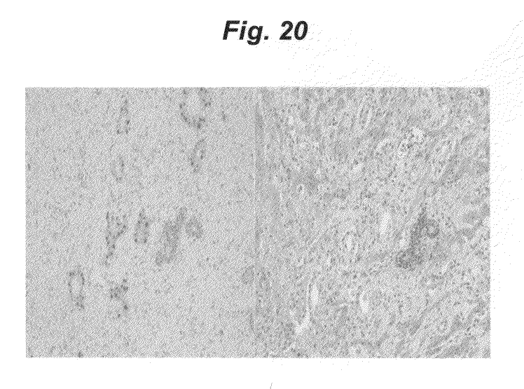

Evaluation of SAKI expression in various cancer cases by immunohistologic staining by using an anti-C-terminal SAKI antibody revealed high expression of SAKI in cancer tissues, independently of cancers, including oral cancer, esophageal cancer, liver cancer, pancreatic cancer, kidney cancer, bladder cancer, ureter cancer, uterine cervix cancer, skin cancer, and mammary gland cancer. The fact indicated that SAKI was useful as a pathological search marker in human cancer diagnosis (FIGS. 19 and 20).

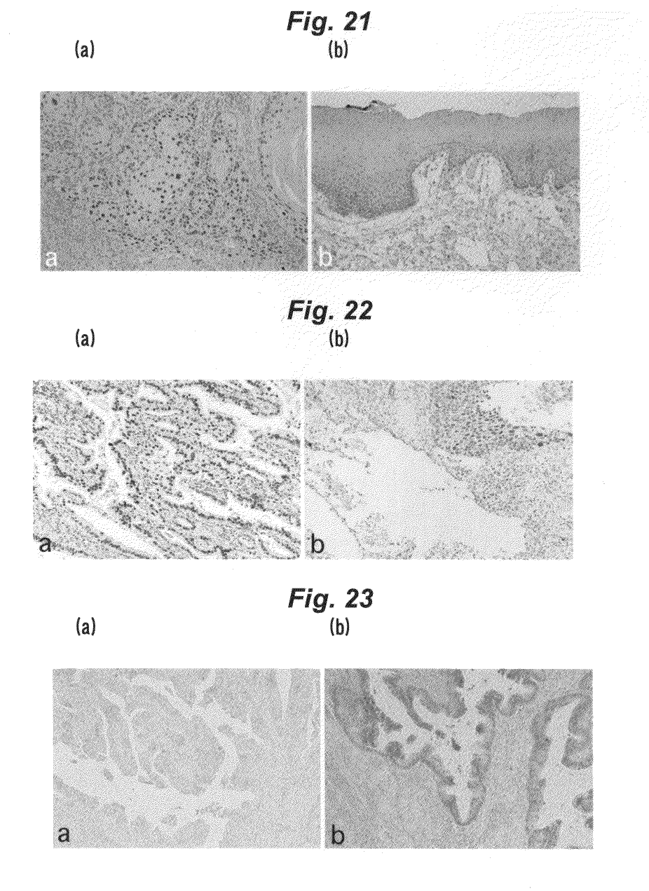

Separately, usefulness of the anti-C-terminal SAKI antibody as a cancer diagnosis marker was studied by examining the difference in staining efficiency between cancer tissues such as oral cancer (squamous cell carcinoma), colon cancer (adenocarcinoma), liver cancer (liver cell carcinoma), lung cancer (squamous cell carcinoma, adenocarcinoma) and prostate cancer (adenocarcinoma) and normal tissues surrounding these cancer tissues (non-cancer tissues) by means of immunohistologic staining by using an anti-C-terminal SAKI antibody. The results are summarized below according to each cancer type. The results obtained by immunohistologic staining of oral and lung cancer tissues are shown respectively in FIGS. 21 and 22.

### (1) Oral Cancer (27 Cases)

As shown in FIG. 21, (a) strong positive reaction was observed in 100.0% (27/27) of oral cavity squamous cell carcinoma tissues. On the other hand, (b) all normal tissues were negative.

### (2) Colon Cancer (19 Cases)

Weak positive reaction was observed in the connective tissue of colon and very weak SAKI reaction in smooth muscle tissue, while strong positive SAKI reaction was observed in 84.2% (16/19) of colon adenocarcinoma tissues.

### (3) Liver Cancer (25 Cases)

Very weak reaction was observed in normal liver tissues such as biliary tract epithelia and pseudobiliary tract, while positive reaction was observed in 72.0% (18/25) of liver cell carcinoma tissues.

### (4) Lung Cancer (Squamous Cell Carcinoma: 8 Cases, and Adenocarcinoma: 13 Cases)

As shown in FIG. 22, (a) distinct positive reaction was observed in 95.2% (20/21) of cancer tissues excluding a single case, independently of whether it is adenocarcinoma or squamous cell carcinoma, while (b) the surrounding pulmonary alveolus and the bronchial tube were negative.

### (5) Prostate Cancer (Adenocarcinoma, 18 Cases)

Very weak SAKI positive reaction was observed in normal tissues such as smooth muscle, skeletal muscle and ganglionic tissue, while medium to strong SAKI positive reaction was observed in the nucleus of adenocarcinoma cells in all cases of cancer tissues.

The results showed that the immunohistologic staining method of using an anti-C-terminal SAKI antibody stained cancer calls at high probability but hardly stained normal cells, which in turn suggested that the anti-C-terminal SAKI antibody was an excellent cancer diagnosis marker.

5-5. Comparison with Conventional Cancer Diagnosis Marker

Expression of SAKI was compared with that of a commonly used pathological diagnostic growth marker Ki-67, by immunohistologic staining of pathological sections of cancer tissue autopsy samples including surrounding normal tissue. As a result, Ki-67 was expressed scatteredly only in cells in proliferation period (FIG. 23(a)), while SAKI was expressed in almost all cancer cells (FIG. 23(b)). The results indicated that SAKI could be used as an indicator of canceration (increase in malignancy) in applications different from those of the traditional cancer markers, such as Ki-67 and PCNA, that have been used as proliferation activity indicators.

### 5-6. Possible Application of the Immunoblotting Method to Cancer Diagnosis

The immunostaining results above showed that SAKI could be a favorable cancer marker. Accordingly, detection of SAKI by Western-blotting method (immunoblotting method) was evaluated, to determine whether the SAKI detection is applicable to actual cancer diagnosis.

The immunoblotting method has a detection sensitivity of dozens to hundred times lower than that of ELISA method (enzyme immunoassay) and of thousand to tens of thousand times lower than that of MASS spectrometry analysis. However, the immunoblotting method allows simple and easy operation in laboratory and also allows detection of immunized protein with confirmation of its molecular weight without construction of an additional special system such as that used in ELISA method.

### (1) Evaluation of Detection Sensitivity by Immunoblotting Method

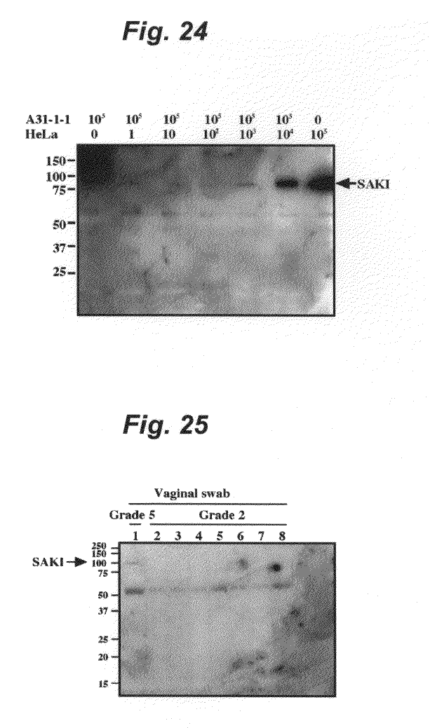

For evaluation of the detection sensitivity by immunoblotting, immunoblotting by using an anti-C-terminal SAKI antibody was carried out, by mixing varying numbers of human uterine cervix cancer cells (HeLa cell) with mouse BALB/c 3T3A31-1-1 cells. The results are summarized in FIG. 24. The amino acid sequence close to the SAKI C-terminal region varies significantly among animal species, and thus, the SAKI in A31-1-1 cell does not cross theoretically.

FIG. 24 shows that contamination by cancer cell can be detected if the cancer cells are contaminated at a rate of 10³ to 10² or more in 10⁵ cells, i.e., if the cancer cell is present at a rate of 1 cell in 100 to 1000 sample cells.

(2) Possibility Detection with Patient Sample

Protein was extracted from the cotton intracelial swab (patient sample) of an uterus cancer patient, and SAKI in the protein was detected by immunoblotting method of using an anti-C-terminal SAKI antibody. The results are summarized in FIG. 25. Eight patient samples of grade-2 uterus cancer (benign but with some abnormal cells), as classified by Japan Association of Obstetricians & Gynecologists, and one patient sample of grade 5 uterus cancer (malignant with atypical cells and with possibility of migration to the surrounding tissue) were used. The amount of the protein used in the immunoblotting method was about 10 μg per lane.

As a result, SAKI was detected only in one case of the cell of grade 5. The fact indicates that the anti-C-terminal SAKI antibody can detect a severe cancer cell with a smaller amount of protein.

### INDUSTRIAL APPLICABILITY

The present invention provides a new substrate protein that is phosphorylated by M-period kinase, and a nucleic acid coding the same, and use of the same. The substrate protein according to the present invention, which was found to be expressed abundantly in various cancer cells, is useful as a cancer diagnosis marker. It is also useful in development of a therapeutic treatment or a therapeutic medicine to cancer. On the other hand, the substrate protein and others or the information (amino acid sequence, nucleotide sequence, etc.) provided by the present invention is useful in studies on the phenomena in which the M-period kinase is involved in a body. Therefore, the disclosure of the present invention would also be useful for elucidation of the biological mechanism in which the M-period kinase is involved and development of therapeutic and diagnostic methods to the diseases in which AIM-1 is involved.

The present invention is not particularly limited by the description in the embodiments and examples above. Various modified embodiments are easily conceived by those who are skilled in the art without departing from the descriptions of the claims, and such modifications are also included in the scope of the present invention.

All disclosure in the papers, patent applications, and patent publications cited above in the present description are incorporated herein by reference in its entirety.

1-14. (canceled)

15. A method of determining a malignancy of an analyte tumor cell, comprising a step of determining an amount of a protein selected from a group consisting of the following proteins (a), (b), (c) and (d) in an analyte tumor cell isolated from a body:
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) a protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase,
wherein a tumor cell in the proliferation period and those in other periods are detected.

16. A method of determining a malignancy of an analyte tumor cell, comprising a step of determining an amount of a protein selected from a group consisting of the following proteins (a), (b), (c) and (d) in an analyte tumor cell isolated from a body:
(a) a protein having the amino acid sequence of SEQ ID No. 2;
(b) a protein having an amino acid sequence partially identical with the amino acid sequence of SEQ ID No. 2 that is a substrate of M-period kinase and has nucleic acid-methylating activity suppressed by phosphorylation;
(c) a protein having the amino acid sequence of SEQ ID No. 2 that is phosphorylated by M-period kinase; and
(d) a protein having a sequence partially different from the amino acid sequence of SEQ ID No. 2 that is phosphorylated by M-period kinase,
wherein cells of a tumor tissue possibly migrating into a surrounding tissue are detected.

17. The method of determining a malignancy of an analyte tumor cell according to claim 15, wherein the amount of the protein is detected by using an immunological staining method.

18. The method of determining a malignancy of an analyte tumor cell according to claim 17, wherein the immunological method is performed by using an antibody to a protein selected from a group consisting of the following proteins (a), (b), (c) and (d):
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) the protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase.

19. An antibody to a protein selected from a group consisting of the following proteins (a), (b), (c) and (d), characterized by being used in the method of determining the malignancy of an analyte tumor cell according to claim 15:
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) a protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase.

20. A method of determining a malignancy of an analyte tumor cell, comprising a step of determining an amount of a nucleic acid selected from a group consisting of the following nucleic acids (A), (B) and (C) in an analyte tumor cell isolated from a body:
(A) a nucleic acid having a nucleotide sequence of SEQ ID No. 1;
(B) a nucleic acid having a nucleotide sequence complementary to the nucleotide sequence of SEQ ID No. 1; and
(C) a nucleic acid hybridizing with a complementary chain of the nucleic acid (A) or (B) under stringent condition and coding a protein that is a substrate of M-period kinase and has nucleic acid-methylating activity suppressed by phosphorylation,
wherein tumor cells in a proliferation period and those in other periods are detected.

21. A method of determining a malignancy of an analyte tumor cell, comprising a step of determining an amount of the nucleic acid selected from a group consisting of the following nucleic acids (A), (B) and (C) in an analyte tumor cell isolated from the body:
(A) a nucleic acid having a nucleotide sequence of SEQ ID No. 1;
(B) a nucleic acid having a nucleotide sequence complementary to the nucleotide sequence of SEQ ID No. 1; and
(C) a nucleic acid hybridizing with a complementary chain of the nucleic acid (A) or (B) under stringent condition and coding a protein that is a substrate of M-period kinase and has nucleic acid-methylating activity suppressed by phosphorylation,
wherein cells of a tumor tissue possibly migrating into a surrounding tissue are detected.

22. A reagent for determining a malignancy of an analyte tumor cell, comprising the antibody according to claim 19, wherein a tumor cell in the proliferation period and those in other periods or alternatively the cells of which the tumor tissue possibly migrates into the surrounding tissue are detected.

23. A reagent for determining a malignancy of an analyte tumor cell, comprising a nucleic acid selected from a group consisting of the following nucleic acids (A), (B) and (C):
(A) a nucleic acid having a nucleotide sequence of SEQ ID No. 1;
(B) a nucleic acid having a nucleotide sequence complementary to the nucleotide sequence of SEQ ID No. 1; and
(C) a nucleic acid hybridizing with a complementary chain of the nucleic acid (A) or (B) under stringent condition and coding a protein that is a substrate of M-period kinase and has nucleic acid-methylating activity suppressed by phosphorylation,
wherein a tumor cell in a proliferation period and those in other periods or alternatively the cells of the tumor tissue possibly migrating into a surrounding tissue are detected.

24. A kit for determining a malignancy of an analyte cell, comprising the reagent according to claim 22 and an instruction manual.

25. The method of determining a malignancy of an analyte tumor cell according to claim 16, wherein the amount of the protein is detected by using an immunological staining method.

26. The method of determining a malignancy of an analyte tumor cell according to claim 25, wherein the immunological method is performed by using an antibody to a protein selected from a group consisting of the following proteins (a), (b), (c) and (d):
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) the protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase.

27. An antibody to a protein selected from a group consisting of the following proteins (a), (b), (c) and (d), characterized by being used in the method of determining the malignancy of an analyte tumor cell according to claim 16:
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) a protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase.

28. An antibody to a protein selected from a group consisting of the following proteins (a), (b), (c) and (d), characterized by being used in the method of determining the malignancy of an analyte tumor cell according to claim 17:
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) a protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase.

29. An antibody to a protein selected from a group consisting of the following proteins (a), (b), (c) and (d), characterized by being used in the method of determining the malignancy of an analyte tumor cell according to claim 25:
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) a protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase.

30. An antibody to a protein selected from a group consisting of the following proteins (a), (b), (c) and (d), characterized by being used in the method of determining the malignancy of an analyte tumor cell according to claim 18:
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) a protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase.

31. An antibody to a protein selected from a group consisting of the following proteins (a), (b), (c) and (d), characterized by being used in the method of determining the malignancy of an analyte tumor cell according to claim 26:
(a) a protein having the amino acid sequence of SEQ ID No. 2;
(b) a protein having an amino acid sequence partially different from the amino acid sequence of SEQ ID No. 2 that is a substrate of M-period kinase and has nucleic acid-methylating activity suppressed by phosphorylation;
(c) a protein having the amino acid sequence of SEQ ID No. 2 that is phosphorylated by M-period kinase; and
(d) a protein having an amino acid sequence partially different from the amino acid sequence of SEQ ID No. 2 that is phosphorylated by M-period kinase.

32. A reagent for determining a malignancy of an analyte tumor cell, comprising the antibody according to claim 27, wherein a tumor cell in the proliferation period and those in other periods or alternatively the cells of which the tumor tissue possibly migrates into the surrounding tissue are detected.

33. A reagent for determining a malignancy of an analyte tumor cell, comprising the antibody according to claim 28, wherein a tumor cell in the proliferation period and those in other periods or alternatively the cells of which the tumor tissue possibly migrates into the surrounding tissue are detected.

34. A reagent for determining a malignancy of an analyte tumor cell, comprising the antibody according to claim 29, wherein a tumor cell in the proliferation period and those in other periods or alternatively the cells of which the tumor tissue possibly migrates into the surrounding tissue are detected.

35. A reagent for determining a malignancy of an analyte tumor cell, comprising the antibody according to claim 30, wherein a tumor cell in the proliferation period and those in other periods or alternatively the cells of which the tumor tissue possibly migrates into the surrounding tissue are detected.

36. A reagent for determining a malignancy of an analyte tumor cell, comprising the antibody according to claim 31, wherein a tumor cell in the proliferation period and those in other periods or alternatively the cells of which the tumor tissue possibly migrates into the surrounding tissue are detected.

37. A kit for determining a malignancy of an analyte cell, comprising the reagent according to claim 32 and an instruction manual.

38. A kit for determining a malignancy of an analyte cell, comprising the reagent according to claim 33 and an instruction manual.

39. A kit for determining a malignancy of an analyte cell, comprising the reagent according to claim 34 and an instruction manual.

40. A kit for determining a malignancy of an analyte cell, comprising the reagent according to claim 35 and an instruction manual.

41. A kit for determining a malignancy of an analyte cell, comprising the reagent according to claim 36 and an instruction manual.

42. A kit for determining a malignancy of an analyte cell, comprising the reagent according to claim 23 and an instruction manual.
